(19) United States
(12) Patent Application Publication
(10) Pub. No.: US 2016/0212997 A1
(43) Pub. Date: Jul. 28, 2016

(54) AGRICULTURAL CHEMICALS

(71) Applicant: REDAG CROP PROTECTION LTD, Manchester (GB)

(72) Inventors: Christopher URCH, Cheshire (GB); William THOMPSON, Hinckley, OH (US)

(21) Appl. No.: 14/917,203

(22) PCT Filed: Sep. 18, 2014
(86) PCT No.: PCT/GB2014/052844

§ 371 Date: Mar. 7, 2016

(30) Foreign Application Priority Data
Sep. 18, 2013 (GB) 1316590.7
Apr. 25, 2014 (GB) 1407329.0

(51) Int. Cl. A01N 43/40; C07D 491/048; C07D 405/04; C07D 263/10; C07D 413/04; C07D 413/12; C07D 405/12; C07D 213/34

(52) U.S. Cl. CPC A01N 43/40; C07D 405/12; C07D 491/048; C07D 213/34; C07D 263/10; C07D 413/04; C07D 413/12; C07D 405/04

(57) ABSTRACT

The present invention relates to compounds which are of use in the field of agriculture as insecticides and acaricides. The compounds contain butenolide rings, oxazoline and isoxazoline rings, pyridine rings or pyranone rings. The invention also relates to compositions comprising said compounds and methods of using said compounds.

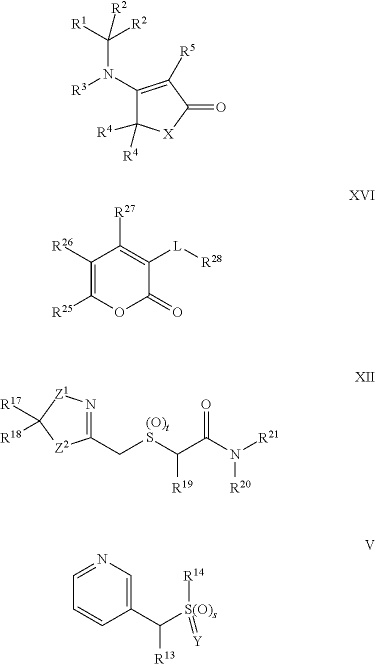

The present invention relates to compounds which are of use in the field of agriculture as insecticides and acaricides.

Given the global increase in demand for food, there is an international need for new treatments to reduce food crop losses to disease, insects and weeds. Over 40% of crops are lost before harvest, and 10% post harvest, worldwide. Losses have actually increased since the mid-1990s.

A new threat contributing to this is the emergence of chemical-resistant organisms, for example, glyphosate-resistant weeds in USA and strobilurin-resistant strains of septoria fungal species.

Recent research also suggests that the geographical spread of many crop pests and diseases is increasing, possibly as a result of global warming.

An aim of the present invention is to provide pesticides (e.g. insecticides and acaricides) which have activity either non-selectively, i.e. broad spectrum activity, or which are active specifically against selective target organisms.

An aim of the present invention is to provide compounds which are less persistent in the environment after use than prior art compounds.

Alternatively or additionally the compounds of the present invention are less prone to bioaccumulation once in the food chain than prior art compounds.

Another aim of the invention is to provide compounds which are less harmful to humans than prior art compounds.

Alternatively or additionally, the compounds of the invention may be less harmful than prior art compounds to one or more of the following groups: amphibians, fish, mammals (including domesticated animals such as dogs, cats, cows, sheep, pigs, goats, etc), reptiles, birds, and beneficial invertebrates (e.g. bees and other insects, or worms), beneficial nematodes, beneficial fungi and nitrogen-fixing bacteria.

The compounds of the invention may be as active or more active than prior art compounds. They may have activity against organisms which have developed a resistance to prior art compounds. However, the present invention also concerns compounds which have a lower level of activity relative to that of prior art compounds. These lower activity compounds are still effective as insecticides and acaricides but have other advantages relative to existing compounds such as, for example, a reduced environmental impact.

The compounds of the invention may be more selective than prior art compounds, i.e. they may have better, similar or even slightly lower activity than the parent against target species but have a significantly lower activity against non-target species (e.g. the crops which are being protected).

This invention provides compounds that achieve one or more of the above aims. The compounds may be active in their own right or may metabolise or react in aqueous media to yield an active compound.

### SUMMARY OF THE INVENTION

Butenolides

In a first aspect of the invention is provided a compound of formula I:

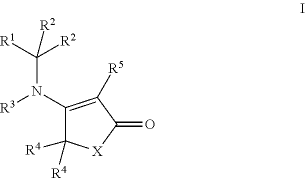

wherein X is independently selected from O or NR⁶;

R¹ is heteroaryl;

R², R⁴ and R⁷ are each independently at each occurrence selected from: H, halogen, C₁-C₄ alkyl and C₁-C₄ haloalkyl;

R³ is independently selected from: (CR⁷R⁷)ₙCO₂R⁸, (CR⁷R⁷)ₙCN, (CR⁷R⁷)ₙCOR⁸, (CR⁷R⁷)ₙCONR⁸R⁸ and (CR⁷CR⁷)CH(OR⁸)₂;

R⁵ is independently selected from H, halogen, C₁-C₄ alkyl and C₁-C₄ haloalkyl; or R³ and R⁵ together with the atoms to which they are attached form a heteroaromatic or heterocycloalkyl ring;

R⁶ an R⁸ are each independently selected from H, C₁-C₄ alkyl, C₃-C₈ cycloalkyl and C₁-C₄ haloalkyl;

n is an integer independently selected from 1, 2 and 3;

wherein in any R¹-R⁸ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}, CO₂R^{a}; C(O)R^{a}; CONR^{a}R^{a}; CH₂NR^{a}R^{a}; CH₂OR^{a} and OR^{a};

wherein R² is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;

or an agronomically acceptable salt or N-oxide thereof.

The compound may be a compound of formula Ia:

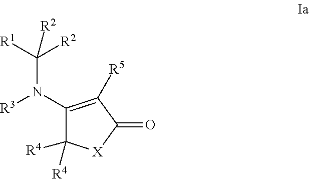

wherein X is independently selected from O or NR⁶;

R¹ is heteroaryl;

R², R⁴ and R⁷ are each independently at each occurrence selected from: H, halogen, C₁-C₄ alkyl and C₁-C₄ haloalkyl;

R³ is (CR⁷R⁷)ₙCO₂R⁸, (CR⁷R⁷)ₙCN, (CR⁷R⁷)ₙCOR⁸, (CR⁷R⁷)ₙCONR⁸R⁸

R⁵ is independently selected from H, halogen, C₁-C₄ alkyl and C₁-C₄ haloalkyl; or R³ and R⁵ together with the atoms to which they are attached form a heteroaromatic ring;

R⁶ an R⁸ are each independently selected from H, C₁-C₄ alkyl, C₃-C₆ cycloalkyl and C₁-C₄ haloalkyl;

n is an integer independently selected from 0, 1, 2 and 3;

wherein in any R¹-R⁸ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a} and OR^{a};

wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;

or an agronomically acceptable salt or N-oxide thereof.

In an embodiment, the compound of formula I or formula Ia is a compound of formula II:

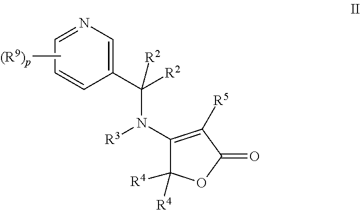

wherein R², R³, R⁴ and R⁵ are as described above for compounds of formula I or formula Ia and wherein R⁹ is independently at each occurrence selected from: C₁-C₄ alkyl, C₁-C₄-haloalkyl, halogen, nitro, OR^{a}, cyano and NR^{a}R^{a}; and p is an integer independently selected from 0, 1, 2, 3 and 4. Alternatively, R⁹ may be selected from: C₁-C₄ alkyl, C₁-C₄-haloalkyl, halogen, nitro, OR¹⁰, SR¹⁰, cyano, C₂-C₄ alkenyl, C₂-C₄ alkynyl, C₃-C₆ cycloalkyl and NR¹⁰R¹⁰; R¹⁰ is independently at each occurrence selected from; H, C₁-C₄ alkyl, C(O)—C₁-C₄-alkyl and C₁-C₄ haloalkyl.

In an embodiment, the compound of formula I or formula Ia is a compound of formula III:

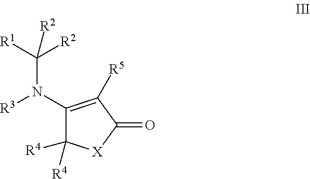

wherein R¹, R², R⁴ and X are as described above for compounds of formula I or formula Ia and wherein R³ is (CR⁷R⁷)ₙCO₂R⁸; and R⁵ is independently selected from H, halogen, C₁-C₄ alkyl and C₁-C₄ haloalkyl.

In an embodiment, the compound of formula I or formula Ia is a compound of formula IV:

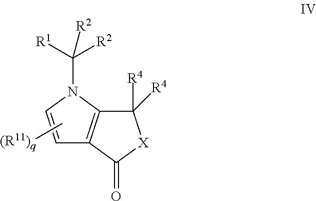

wherein R¹, R², R⁴ and X are as described above for compounds of formula I or formula Ia and wherein R¹¹ is independently at each occurrence selected from: C₁-C₄ alkyl, C₁-C₄-haloalkyl, halogen, nitro, OR^{a}, cyano and NR^{a}R^{a}; and q is an integer independently selected from 0, 1 and 2. Alternatively, R¹¹ may be selected from: C₁-C₄ alkyl, C₁-C₄-haloalkyl, halogen, nitro, OR¹⁰, SR¹⁰, cyano, C₂-C₄ alkenyl, C₂-C₄ alkynyl, C₃-C₆ cycloalkyl and NR¹⁰R¹⁰, R¹⁰ is independently at each occurrence selected from; H, C₁-C₄ alkyl, C(O)—C₁-C₄-alkyl and C₁-C₄ haloalkyl.

The following embodiments apply to compounds of any of formulae (I)-(IV) (including formula Ia). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

In an embodiment, n is an integer independently selected from 1, 2 and 3. Preferably, n is 1.

In an embodiment, X is NR⁶. R⁶ may be H. Thus X may be NH. Preferably, X is O.

In an embodiment, R¹ is a 6-membered heterocycle. Thus, R¹ may be pyridine, pyrimidine, pyrazine or pyridazine. Preferably, R′ is pyridine. Preferably R¹ represents

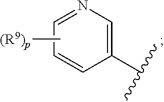

wherein R⁹ is independently at each occurrence selected from: C₁-C₄ alkyl, C₁-C₄-haloalkyl, halogen, nitro, OR^{a}, cyano and NRaRa; and p is an integer independently selected from 0, 1, 2, 3 and 4. Alternatively, R⁹ may be selected from: C₁-C₄ alkyl, C₁-C₄-haloalkyl, halogen, nitro, OR¹⁰, SR¹⁰, cyano, C₂-C₄ alkenyl, C₂-C₄ alkynyl, C₃-C₆ cycloalkyl and NR¹⁰R¹⁰; R¹⁰ is independently at each occurrence selected from; H, C₁-C₄ alkyl, C(O)—C₁-C₄-alkyl and C₁-C₄ haloalkyl. In an embodiment, p is 1. Thus, R¹ may represent

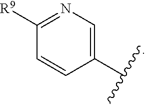

In an embodiment, R⁹ is selected from C₁-C₄ alkyl, C₁-C₄-haloalkyl and halogen. R⁹ may also be C₃-C₆-cycloalkyl. Preferably R⁹ is halogen, e.g. chloride. Alternatively, R⁹ can be C₁-C₄ haloalkyl, e.g. CF₃. In a specific embodiment, R¹ represents

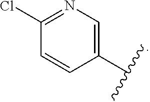 [SMILES: *c1ccc(Cl)nc1]

Preferably, R² is at each occurrence H.

It may be that R³ is selected from: (CR⁷R⁷)ₙCO₂R⁸, (CR⁷R⁷)ₙCN, (CR⁷R⁷)ₙCOR⁸ and (CR⁷R⁷)ₙCONR⁸R⁸.

In an embodiment, R³ is (CR⁷R⁷)ₙCO₂R⁸. In an embodiment, n an integer independently selected from 1, 2 and 3. In an embodiment, n is 1. In another embodiment, R⁷ is at each occurrence H. Thus, R³ may be CH₂CO₂R⁸. In a specific embodiment, R⁸ is C₁-C₄ alkyl, e.g. ethyl.

In an embodiment, R⁵ is H.

In an embodiment, R³ and R⁵ together with the atoms to which they are attached form a heteroaromatic ring (e.g. a 5-membered heteroaromatic ring). In a further embodiment, R³ and R⁵ together with the atoms to which they are attached form a pyrrole ring. In yet another embodiment, the pyrrole ring is unsubstituted (i.e. unsubstituted other than the groups already depicted in formula I).

In an embodiment, q is 0.

Preferably, R⁴ is at each occurrence H.

Pyridinyl Sulphur Compounds

In a second aspect of the invention is provided a compound of formula V:

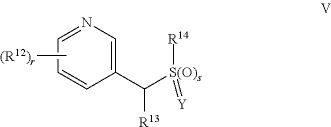

wherein Y is independently selected from N—S(O)₂R¹⁵, N—C(O)R¹⁵, NC(O)OR¹⁵, NC(O)NR^{15a}R¹⁵; R¹² is independently at each occurrence selected from: C₁-C₄ alkyl, C₁-C₄-haloalkyl, halogen, nitro, OR¹⁶, SR¹⁶, cyano, C₂-C₄ alkenyl, C₂-C₄ alkynyl, C₃-C₆ cycloalkyl and NR¹⁶R¹⁶; R¹⁶ is independently at each occurrence selected from; H, C₁-C₄ alkyl, C(O)—C₁-C₄-alkyl and C₁-C₄ haloalkyl;

R¹³ is independently selected from: H, C₁-C₄ alkyl and C₁-C₄ haloalkyl;

R¹⁴ and R¹⁵ are each independently selected from: aryl, heteroaryl, C₁-C₄ alkyl and C₁-C₄ haloalkyl;

R^{15a} is independently selected from: H, C₁-C₄ alkyl and C₁-C₄ haloalkyl;

r is an integer independently selected from 0, 1, 2, 3 and 4;

s is an integer selected from 0 and 1;

wherein in any R¹²-R¹⁶ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂Ra; SO₂NR^{a}R^{a}; CO₂R^{a}; C(O)R^{a}; CONR^{a}R^{a}; CH₂NR^{a}R^{a}; CH₂OR^{a} and OR^{a};

wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;

or an agronomically acceptable salt or N-oxide thereof.

The compound may be a compound of formula Va:

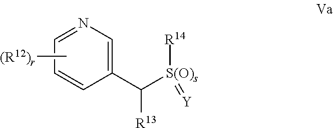

wherein Y is independently selected from O, N—S(O)₂R¹⁵, N—CN, N—C(O)R¹⁵, NC(O)OR¹⁵ NC(O)NR¹⁵R¹⁵;

R¹² is independently at each occurrence selected from: C₁-C₄ alkyl, C₁-C₄-haloalkyl, halogen, nitro, OR¹⁶, SR¹⁶, cyano, C₂-C₄ alkenyl, C₂-C₄ alkynyl, C₃-C₆ cycloalkyl and NR¹⁶R¹⁶; R¹⁶ is independently at each occurrence selected from; H, C₁-C₄ alkyl, C(O)—C₁-C₄-alkyl and C₁-C₄ haloalkyl;

R¹³ is independently selected from: H, C₁-C₄ alkyl and C₁-C₄ haloalkyl;

R¹⁴ and R¹⁵ are each independently at each occurrence selected from: C₁-C₄ alkyl and C₁-C₄ haloalkyl;

r is an integer independently selected from 0, 1, 2, 3 and 4;

s is an integer selected from 0 and 1; with the proviso that if Y is N—CN, s is 0;

wherein in any R¹²-R¹⁶ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a} and OR^{a};

wherein R^{a} is selected from H, C₁-C₄ alkyl and C₁-C₄ haloalkyl; and wherein, in the case of an aryl group or heteroaryl group, any two of these substituents (e.g. NR²R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;

or an agronomically acceptable salt or N-oxide thereof.

In a third aspect of the invention is provided a method of controlling insect and arachnid pests of plants, the method comprising applying an agronomically effective and substantially non-phytotoxic (to the crop plant) quantity of a compound of formula Va or formula Vb to the seeds of the plants, to the plants themselves or to the area where it is intended that the plants will grow;

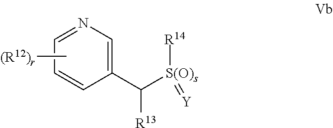

wherein Y is independently selected from O and N—CN;

R¹² is independently at each occurrence selected from: C₁-C₄ alkyl, C₁-C₄-haloalkyl, halogen, nitro, OR¹⁶, SR¹⁶, cyano, C₂-C₄ alkenyl, C₂-C₄ alkynyl, C₃-C₆ cycloalkyl and NR¹⁶R¹⁶; R¹⁶ is independently at each occurrence selected from; H, C₁-C₄ alkyl, C(O)—C₁-C₄-alkyl and C₁-C₄ haloalkyl;

R¹³ is independently selected from: H, C₁-C₄ alkyl and C₁-C₄ haloalkyl;

R¹⁴ is independently selected from: aryl, heteroaryl, C₃-C₈-cycloalkyl, C₃-C₈-heterocycloalkyl, C₁-C₄ alkyl and C₁-C₄ haloalkyl;

r is an integer independently selected from 0, 1, 2, 3 and 4;

s is an integer selected from 0 and 1; with the proviso that if Y is N—CN, s is 0;

wherein in any R¹²-R¹⁶ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a} and OR^{a};

wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;

or an agronomically acceptable salt or N-oxide thereof.

In a fourth aspect of the invention is provided an insecticidal or acaricidal composition comprising an effective and non-phytotoxic amount of a compound of formula Va or formula Vb.

In an embodiment, the compound of formula V or formula Vb is a compound of formula VI:

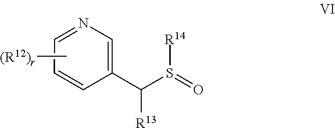

wherein R¹², R¹³, R¹⁴ and r are as described above for compounds of formula V or formula Vb.

In an embodiment, the compound of formula V or formula Vb is a compound of formula VII:

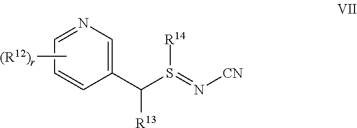

wherein R¹², R¹³, R¹⁴ and r are as described above for compounds of formula V or formula Vb.

In an embodiment, the compound of formula V or formula Va is a compound of formula VIII:

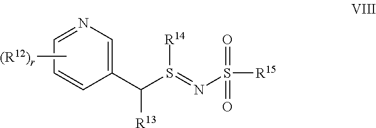

wherein R¹², R¹³, R¹⁴, R¹⁵ and r are as described above for compounds of formula V or formula Va.

In an embodiment, the compound of formula V or formula Va is a compound of formula IX:

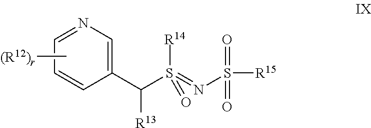

wherein R¹², R¹³, R¹⁴, R¹⁵ and r are as described above for compounds of formula V or formula Va.

In an embodiment, the compound of formula V or formula Va is a compound of formula X:

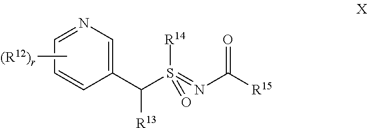

wherein R¹², R¹³, R¹⁴, R¹⁵ and r are as described above for compounds of formula V or formula Va. Preferably, R¹⁵ is unsubstituted C₁-C₃ alkyl.

In an embodiment, the compound of formula V or formula Va is a compound of formula XI:

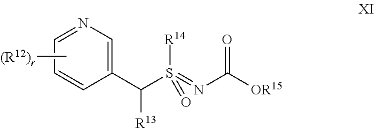

wherein R¹², R¹³, R¹⁴, R¹⁵ and r are as described above for compounds of formula V or formula Va. Preferably, R¹⁵ is unsubstituted C₁-C₃ alkyl.

The following embodiments apply to compounds of any of formulae (V)-(XI) (including Va and Vb). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

In an embodiment, r is 1. In an embodiment, the pyridine ring to which the R¹² group is attached takes the form

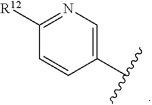

In an embodiment, R¹² is selected from C₁-C₄ alkyl, C₁-C₄-haloalkyl, halogen and C₃-C₆-cycloalkyl. In another embodiment, R¹² is C₁-C₄ haloalkyl, e.g. CF₃. Alternatively, R¹² may be halogen, e.g. Cl. In a specific embodiment, the pyridine to which the R¹² group is attached takes the form

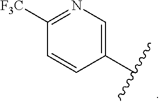 [SMILES: *c1ccc(C(F)(F)F)nc1]

In an embodiment, R¹³ is C₁-C₄ alkyl. Preferably R¹³ is methyl.

R¹⁴ may be selected from C₁-C₄ alkyl and C₁-C₄ haloalkyl. In an embodiment, R¹⁴ is C₁-C₄ alkyl. Preferably R¹⁴ is methyl.

R¹⁵ may be selected from C₁-C₄ alkyl and C₁-C₄ haloalkyl. In an embodiment, R¹⁵ is C₁-C₄ alkyl. R¹⁵ may be unsubstituted C₁-C₃ alkyl. Preferably R¹⁵ is methyl.

Alternatively, R¹⁵ may be aryl or heteroaryl. In a further alternative, R¹⁵ may be selected from C₃-C₈-cycloalkyl, C₃-C₈-heterocycloalkyl.

It may be that R¹⁵ is independently selected from: aryl, heteroaryl, C₃-C₈-cycloalkyl, C₃-C₈-heterocycloalkyl, C₁-C₄ alkyl and C₁-C₄ haloalkyl; and R¹⁴ is independently selected from C₁-C₄ alkyl and C₁-C₄ haloalkyl.

R^{15a} may be selected from C₁-C₄ alkyl and C₁-C₄ haloalkyl. Preferably, however, R^{15a} is H.

In an embodiment, s is 0. Alternatively, s may be 1.

In an embodiment, Y is independently selected from N—S(O)₂R¹⁵, N—CN, N—C(O)R¹⁵, NC(O)OR¹⁵. In another embodiment, Y is independently selected from O, N—S(O)₂R¹⁵, NC(O)R¹⁵, NC(O)OR¹⁵. In yet another embodiment, Y is independently selected from N—S(O)₂R¹⁵, NC(O)R¹⁵, NC(O)OR¹⁵. In a specific embodiment, Y is O. In another specific embodiment, Y is N—S(O)₂R¹⁵, e.g. N—S(O)₂Me. In yet another specific embodiment, Y is N—CN. In a yet further specific embodiment, Y is selected from N—C(O)R¹⁵ and NC(O)OR¹⁵. Thus, Y may be N—C(O)R¹⁵, e.g. N—C(O)Me. Y may also be NC(O)OR¹⁵, e.g. NC(O)OMe.

Y may be independently selected from N—CN, N—S(O)₂R¹⁵, N—C(O)R¹⁵, NC(O)NR^{15a}R¹⁵. Preferably, if Y is selected from N—CN or NC(O)OR¹⁵, s is 0. Thus, it may be that if Y is NC(O)OR¹⁵, s is 0.

It may be that if Y is NC(O)OR¹⁵ the compound is not an N-oxide.

Where Y is N—S(O)₂R¹⁵, preferably s is 1. Where Y is N—S(O)₂R¹⁵, R¹⁵ may be aryl or heteroaryl. Alternatively, where Y is N—S(O)₂R¹⁵, R¹⁵ may be selected from C₁-C₄ alkyl and C₁-C₄ haloalkyl. In a further alternative, where Y is N—S(O)₂R¹⁵, R¹⁵ may be selected from C₃-C₈-cycloalkyl, C₃-C₈-heterocycloalkyl.

Preferably, where Y is N—C(O)R¹⁵, R¹⁵ is unsubstituted C₁-C₃ alkyl.

Oxazolines and Isoxazolines

In a fourth aspect of the invention is provided a compound of formula XII:

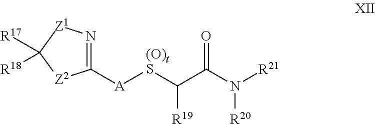

wherein Z¹ and Z² are each selected from O and CH₂; with the proviso that one of Z¹ and Z² is O and the other is CH₂;

A is independently selected from a phenyl group, a pyridyl group, a pyridazyl group, a pyrimidyl group, a pyrazyl group and a thiophenyl group;

R¹⁷ is aryl;

R¹⁸ is independently selected from H, halogen, C₁-C₄ alkyl and C₁-C₄ haloalkyl;

R¹⁹ is independently selected from H, halogen, C₁-C₄ alkyl, C₃-C₆-cycloalkyl and C₁-C₄ haloalkyl;

R²⁰ and R²¹ are each independently selected from H, C₁-C₄ alkyl, C₃-C₆-cycloalkyl and C₁-C₄ haloalkyl; or wherein R¹⁹ and R²⁰, together with the atoms to which they are attached form a 5- or 6-membered lactam ring; with the proviso that, if Z¹ is O and R¹⁹ and R²⁰ do not, together with the atoms to which they are attached, form a lactam ring, A is thiophenyl;

t is an integer independently selected from 0, 1 and 2;

wherein in any R¹⁷-R²¹ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a} and OR^{a};

wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;

or an agronomically acceptable salt or N-oxide thereof.

In an embodiment, the compound of formula XII is a compound of formula XIII:

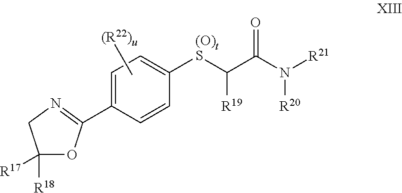

wherein R¹⁷, R¹⁸, R¹⁹, R²⁰, R²¹, and t are as described above for compounds of formula XII and wherein R¹⁹ is selected from H, halogen, C₁-C₄ alkyl, C₃-C₆-cycloalkyl and C₁-C₄ haloalkyl; R²⁰ is independently selected from H, C₁-C₄ alkyl, C₃-C₆-cycloalkyl and C₁-C₄ haloalkyl; R²² is independently at each occurrence selected from: C₁-C₄ alkyl, C₁-C₄-haloalkyl, halogen, nitro, OR^{a}, cyano and NR^{a}R^{a}; and u is an integer independently selected from 0, 1, 2, 3 and 4. It may be that R²² is selected from: C₁-C₄ alkyl, C₁-C₄-haloalkyl, halogen, nitro, OR²³, SR²³, cyano, C₂-C₄ alkenyl, C₂-C₄ alkynyl, C₃-C₆ cycloalkyl and NR²³R²³; R²³ is independently at each occurrence selected from; H, C₁-C₄ alkyl, C(O)—C₁-C₄-alkyl and C₁-C₄ haloalkyl.

In an embodiment, the compound of formula XII is a compound of formula XIV:

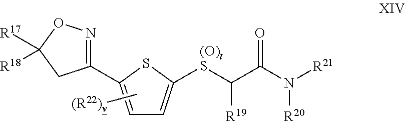

wherein R¹⁷, R¹⁸, R²¹ and t are as described above for compounds of formula XII; wherein R²² is as described above for compounds of formula XIII; and wherein R¹⁹ is selected from H, halogen, C₁-C₄ alkyl, C₃-C₆-cycloalkyl and C₁-C₄ haloalkyl;

R²⁹ is independently selected from H, C₁-C₄ alkyl, C₃-C₆-cycloalkyl and C₁-C₄ haloalkyl; and v is an integer independently selected from 0, 1 and 2.

In an embodiment, the compound of formula XII is a compound of formula XV:

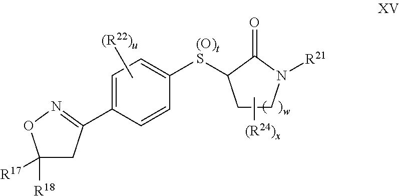

wherein R¹⁷, R¹⁸, R²¹, and t are as described above for compounds of formula XII; wherein R²² and u are as described above for compounds of formula XIII; and wherein R²⁴ is independently at each occurrence selected from: oxo; ═NR^{a}; ═NOR^{a}; halogen, OR^{a}, NR^{a}R^{a}, C₁-C₄ alkyl, and C₁-C₄ haloalkyl; w is an integer independently selected from 1 and 2; and x is an integer selected from 0, 1, 2, 3, 4, 5 and 6. It may also be that R²⁴ is selected from halogen, C₁-C₄ alkyl, C₃-C₆ cycloalkyl and C₁-C₄ haloalkyl. For the absence of doubt, where w is 1, x is independently selected from 0, 1, 2, 3 and 4.

The following embodiments apply to compounds of any of formulae (XII)-(XV). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

In an embodiment, t is 0. In another embodiment, t is 1.

In an embodiment, Z¹ is O and Z² is CH₂. In another embodiment, Z¹ is CH₂ and Z² is O.

R¹⁷ may be unsubstituted phenyl or phenyl substituted with from 1 to 4 substituents selected from C₁-C₄ alkyl, C1-C4-haloalkyl, halogen, nitro, OR²³, SR²³, cyano, C₂-C₄ alkenyl, C₂-C₄ alkynyl, C₃-C₆ cycloalkyl and NR²³R²³. R¹⁷ may be substituted with from 1 to 4 substituents selected from C₁-C₄ alkyl, C₁-C₄-haloalkyl, halogen, nitro, OR^{a}, cyano and NR^{a}R^{a}. In an embodiment, R¹⁷ is a phenyl group substituted with from 1 to 4 substituents selected from C₁-C₄ alkyl, C₁-C₄-haloalkyl, halogen and C₃-C₆ cycloalkyl. R¹⁷ may be a phenyl group substituted with from 1 to 4 substituents selected from C₁-C₄ alkyl, C₁-C₄-haloalkyl and halogen. In an embodiment, R¹⁷ is a phenyl group substituted with from 1 to 4 (e.g. 2) halogen substituents. Said halogen substituents may be the same or different. If, for example, the phenyl has two halogen substituents they may both be Cl. As another example, if the phenyl has three halogen substituents, it may be that two are Cl and the third may be F. In a specific embodiment, R¹⁷ is

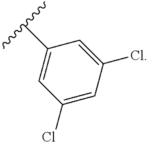 [SMILES: *c1cc(Cl)cc(Cl)c1]

In an embodiment, R¹⁸ is C₁-C₄ haloalkyl. Preferably, R¹⁸ is CF₃.

In an embodiment, A is phenyl. Thus, A may represent

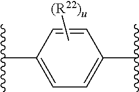

wherein R²² is independently at each occurrence selected from: C₁-C₄ alkyl, C₁-C₄-haloalkyl, halogen, nitro, OR^{a}, cyano and NR^{a}R^{a} and u is an integer independently selected from 0, 1, 2, 3 and 4. It may also be that R²² is independently at each occurrence selected from: C₁-C₄ alkyl, C₁-C₄-haloalkyl, halogen, nitro, OR²³, SR²³, cyano, C₂-C₄ alkenyl, C₂-C₄ alkynyl, C₃-C₆ cycloalkyl and NR²³R²³; and R²³ is independently at each occurrence selected from; H, C₁-C₄ alkyl, C(O)—C₁-C₄-alkyl and C₁-C₄ haloalkyl. In an embodiment, u is 1. Thus, A may represent

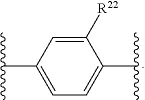

In an embodiment, R²² is independently at each occurrence selected from C₁-C₄ alkyl, C₁-C₄-haloalkyl and halogen. R²² may therefore be at one occurrence C₁-C₄ alkyl, e.g. methyl. In a specific embodiment, A is

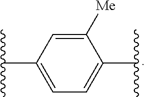 [SMILES: *c1ccc(*)c(C)c1]

In another embodiment, A is thiophenyl. Thus, A may represent

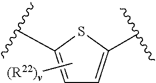

wherein R²² is independently at each occurrence selected from: C₁-C₄ alkyl, C₁-C₄-haloalkyl, halogen, nitro, OR^{a}, cyano and NR^{a}R^{a} and v is an integer independently selected from 0, 1 and 2. It may be that R²² is independently at each occurrence selected from: C₁-C₄ alkyl, C₁-C₄-haloalkyl, halogen, nitro, OR²³, SR²³, cyano, C₂-C₄ alkenyl, C₂-C₄ alkynyl, C₃-C₆ cycloalkyl and NR²³R²³; and R²³ is independently at each occurrence selected from; H, C₁-C₄ alkyl, C(O)—C₁-C₄-alkyl and C₁-C₄ haloalkyl. In an embodiment, v is 1. Thus, A may represent

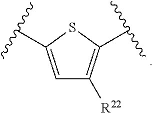

In an embodiment, R²² is independently at each occurrence selected from C₁-C₄ alkyl, C₁-C₄-haloalkyl and halogen. R²² may therefore be at one occurrence C₁-C₄ alkyl, e.g. methyl. In a specific embodiment, A is

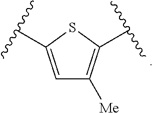 [SMILES: *c1cc(C)c(*)s1]

In another embodiment, v is 0. Thus, in another specific embodiment, A is

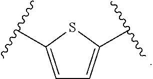 [SMILES: *c1ccc(*)s1]

In an embodiment, R¹⁹ is H. In an embodiment, R²⁰ is H. In an embodiment, R¹⁹ and R²⁰ are each H.

In an embodiment, R¹⁹ and R²⁰, together with the atoms to which they are attached form a 5- or 6-membered lactam ring, e.g. a 5-membered lactam ring. Thus, the portion of the molecule to the right (as drawn in formula XI above) of the S(O)ₜ group may be

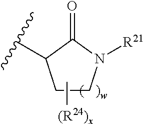

wherein R²⁴ is independently at each occurrence selected from: oxo; ═NR^{a}; ═NOR^{a}; halogen, OR^{a}, NR^{a}R^{a}, C₁-C₄ alkyl, and C₁-C₄ haloalkyl; w is an integer independently selected from 1 and 2; and x is an integer selected from 0, 1, 2, 3, 4, 5 and 6. It may also be that R²⁴ is independently at each occurrence selected from halogen, C₁-C₄ alkyl, C₃-C₈-cycloalkyl and C₁-C₄ haloalkyl. Preferably, w is 1. Preferably, x is 0. Thus, the portion of the molecule to the right (as drawn in formula XI above) of the S(O)ₜ group may be

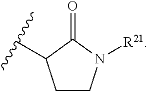

In an embodiment, R²¹ is H. In another embodiment, R²¹ is C₁-C₄ haloalkyl, e.g. CH₂CF₃.

Pyranones

In a fifth aspect of the invention is provided a compound of formula XVI:

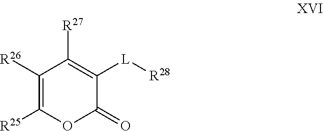

wherein L is independently selected from —NR²⁹—C(O)— and —N═CR³⁰—;

R²⁵ is independently selected from pyridyl, pyrimidyl, pyrazyl and pyridazyl;

R²⁶ and R²⁷ are each independently selected from H, halogen, C₁-C₄ alkyl and C₁-C₄ haloalkyl;

R²⁹ is independently selected from H, OR³¹, C₁-C₄ alkyl, C₃-C₆ cycloalkyl and C₁-C₄ haloalkyl;

R²⁸, R³⁰ and R³¹ are each independently selected from H, C₁-C₄ alkyl, C₃-C₆ cycloalkyl and C₁-C₄ haloalkyl; or R²⁷ and R²⁸, together with the atoms to which they are attached, form a 5- or 6-membered lactam ring;

with the proviso that if R²⁵ is pyridyl, L is —NR²⁹—C(O)— and R²⁷ and R²⁸ do not, together with the atoms to which they are attached, form a 5- or 6-membered lactam ring, R²⁹ is OR³¹;

wherein in any R²⁵-R³¹ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a} and OR^{a};

wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;

or an agronomically acceptable salt or N-oxide thereof.

For the absence of doubt, if R²⁷ and R²⁸, together with the atoms to which they are attached, form a 5- or 6-membered lactam ring, L must be —NR²⁹—C(O)—.

Where L is described as being —NR²⁹—C(O)— it is intended that the nitrogen atom of the NR²⁹ portion of L is directly bonded to the pyranone ring of the compound of the invention and the carbon atom of the C(O) portion of L is directly bonded to the R²⁸ group. Likewise, where L is described as being —N═CR³⁰— it is intended that the nitrogen atom of L is directly bonded to the pyranone ring of the compound of the invention and the carbon atom of the CR³⁰ portion of L is directly bonded to the R²⁸ group.

In an embodiment, the compound of formula XVI is a compound of formula XVII:

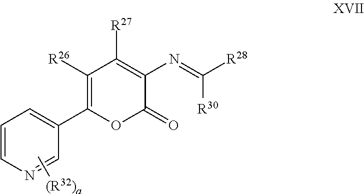

wherein R²⁶, R²⁷, R²⁸ and R³⁰ are as described above for compounds of formula XVI and wherein R²⁷ is independently selected from H, halogen, C₁-C4 alkyl and C₁-C4 haloalkyl; R²⁸ is independently selected from H, C₁-C4 alkyl, C₃-C₆ cycloalkyl and C₁-C₄ haloalkyl; R³² is independently at each occurrence selected from: C₁-C₄ alkyl, C₁-C₄-haloalkyl, halogen, nitro, OR^{a}, cyano and NR^{a}R^{a}; and a is an integer independently selected from 0, 1, 2, 3 and 4. It may be that R³² is independently at each occurrence selected from: C₁-C₄ alkyl, C₁-C₄-haloalkyl, halogen, nitro, OR³³, SR³³, cyano, C₂-C₄ alkenyl, C₂-C₄ alkynyl, C₃-C₆ cycloalkyl and NR³³R³³; and R³³ is independently at each occurrence selected from; H, C₁-C₄ alkyl, C(O)—C₁-C₄-alkyl and C₁-C₄ haloalkyl.

In an embodiment, the compound of formula XVI is a compound of formula XVIII:

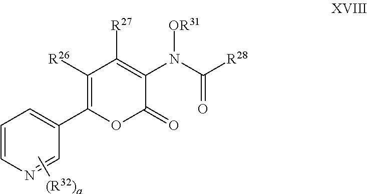

wherein R²⁶ and R³¹ are as described above for compounds of formula XVI and wherein R²⁷ is independently selected from H, halogen, C₁-C₄ alkyl and C₁-C₄ haloalkyl; R²⁸ is independently selected from H, C₁-C₄ alkyl, C₃-C₆ cycloalkyl and C₁-C₄ haloalkyl; R³² is independently at each occurrence selected from: C₁-C₄ alkyl, C₁-C₄-haloalkyl, halogen, nitro, OR^{a}, cyano and NR^{a}R^{a}; and a is an integer independently selected from 0, 1, 2, 3 and 4. It may be that R³² is independently at each occurrence selected from: C₁-C₄ alkyl, C₁-C₄-haloalkyl, halogen, nitro, OR³³, SR³³, cyano, C₂-C₄ alkenyl, C₂-C₄ alkynyl, C₃-C₆ cycloalkyl and NR³³R³³; and R³³ is independently at each occurrence selected from; H, C₁-C₄ alkyl, C(O)—C₁-C₄-alkyl and C₁-C₄ haloalkyl.

In an embodiment, the compound of formula XVI is a compound of formula XIX:

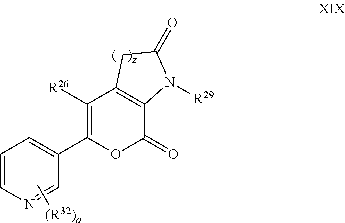

wherein R²⁵, R²⁶ and R²⁹ are as described above for compounds of formula XVI and wherein R²⁹ is independently selected from H, C₁-C₄ alkyl, C₃-C₆ cycloalkyl and C₁-C₄ haloalkyl; R³² is independently at each occurrence selected from: C₁-C₄ alkyl, C₁-C₄-haloalkyl, halogen, nitro, OR^{a}, cyano and NR^{a}R^{a}; a is an integer independently selected from 0, 1, 2, 3 and 4; and z is an integer independently selected from 1 and 2. It may be that R³² is independently at each occurrence selected from: C₁-C₄ alkyl, C₁-C₄-haloalkyl, halogen, nitro, OR³³, SR³³, cyano, C₂-C₄ alkenyl, C₂-C₄ alkynyl, C₃-C₆ cycloalkyl and NR³³R³³; and R³³ is independently at each occurrence selected from; H, C₁-C₄ alkyl, C(O)—C₁-C₄-alkyl and C₁-C₄ haloalkyl.

In an embodiment, the compound of formula XVI is a compound of formula XX:

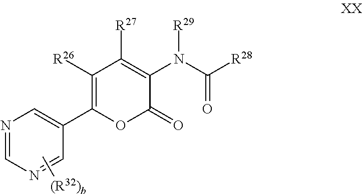

wherein R²⁶ is as described above for compounds of formula XVI and wherein R²⁷ is independently selected from H, halogen, C₁-C₄ alkyl and C₁-C₄ haloalkyl; R²⁸ and R²⁹ are each independently selected from H, C₁-C₄ alkyl, C₃-C₆ cycloalkyl and C₁-C₄ haloalkyl; R³² is independently at each occurrence selected from: C₁-C₄ alkyl, C₁-C₄-haloalkyl, halogen, nitro, OR^{a}, cyano and NR^{a}R^{a}; and b is an integer independently selected from 0, 1, 2 and 3. It may be that R³² is independently at each occurrence selected from: C₁-C₄ alkyl, C₁-C₄-haloalkyl, halogen, nitro, OR³³, SR³³, cyano, C₂-C₄ alkenyl, C₂-C₄ alkynyl, C₃-C₆ cycloalkyl and NR³³R³³; and R³³ is independently at each occurrence selected from; H, C₁-C₄ alkyl, C(O)—C₁-C₄-alkyl and C₁-C₄ haloalkyl.

The following embodiments apply to compounds of any of formulae (XVI)-(XX). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

In an embodiment, R²⁵ is pyridyl, e.g. 3-pyridyl. Thus R²⁵ may be

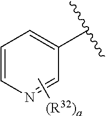

wherein R³² is independently at each occurrence selected from: C₁-C₄ alkyl, C₁-C₄-haloalkyl, halogen, nitro, OR^{a}, cyano and NR^{a}R^{a}; and a is an integer independently selected from 0, 1, 2, 3 and 4. It may be that R³² is independently at each occurrence selected from: C₁-C₄ alkyl, C₁-C₄-haloalkyl, halogen, nitro, OR³³, SR³³, cyano, C₂-C₄ alkenyl, C₂-C₄ alkynyl, C₃-C₆ cycloalkyl and NR³³R³³; and R³³ is independently at each occurrence selected from; H, C₁-C₄ alkyl, C(O)—C₁-C₄-alkyl and C₁-C₄ haloalkyl. Preferably, a is 0. Thus R²⁵ may be unsubstituted pyridyl, e.g. unsubstituted 3-pyridyl.

In an embodiment, R²⁵ is independently selected from pyrimidyl, pyrazyl and pyridazyl. R²⁵ may be pyrimidyl. Thus, R²⁵ may be

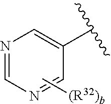

wherein R³² is independently at each occurrence selected from: C₁-C₄ alkyl, C₁-C₄-haloalkyl, halogen, nitro, OR^{a}, cyano and NR^{a}R^{a}; and b is an integer independently selected from 0, 1, 2 and 3. It may be that R³² is independently at each occurrence selected from: C₁-C₄ alkyl, C₁-C₄-haloalkyl, halogen, nitro, OR³³, SR³³, cyano, C₂-C₄ alkenyl, C₂-C₄ alkynyl, C₃-C₆ cycloalkyl and NR³³R³³; R³³ is independently at each occurrence selected from; H, C₁-C₄ alkyl, C(O)—C₁-C₄-alkyl and C₁-C₄ haloalkyl. Preferably, b is 0. Thus, R²⁵ may be unsubstituted pyrimidyl, e.g.

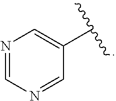 [SMILES: *c1cncnc1]

Preferably, R²⁶ is H.

In an embodiment, R²⁷ is H.

In an embodiment, R²⁸ is independently selected from C₁-C₄ alkyl, C₃-C₆ cycloalkyl and C₁-C₄ haloalkyl. In an embodiment, R²⁸ is C₁-C₄ alkyl or C₃-C₆ cycloalkyl. In a specific embodiment, R²⁸ is cyclopropyl.

Alternatively, R²⁷ and R²⁸, together with the atoms to which they are attached, form a 5- or 6-membered lactam ring. In an embodiment, R²⁷ and R²⁸, together with the atoms to which they are attached, form a 5-membered lactam ring. Thus, R²⁷ and R²⁸ together with L and the respective pyranone atoms form

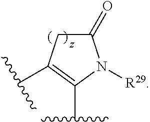

In an embodiment, R²⁹ is H. In an embodiment, z is 1. Thus, in a specific embodiment, R²⁷ and R²⁸ together with L and the respective pyranone atoms form

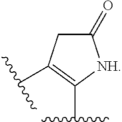 [SMILES: *C1=C(*)NC(=O)C1]

Alternatively, Z may be 2. Thus, in another specific embodiment, R²⁷ and R²⁸ together with L and the respective pyranone atoms form

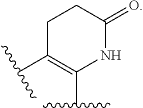 [SMILES: *C1=C(*)NC(=O)CC1]

In an embodiment, L is —NR²⁹—C(O)—. In an embodiment, R²⁹ is selected from H, C₁-C₄ alkyl, C₃-C₆ cycloalkyl and C₁-C₄ haloalkyl. Specifically, R²⁹ may be H. Thus, L may be —NH—C(O)—. In another embodiment, R²⁹ is OR³¹. In a further embodiment, R³¹ is H. Thus, L may be —N(OH)—C(O)—.

Alternatively, L is —N═CR³⁰—. Preferably R³⁰ is H. Thus, L may be —N═CH—.

The following compounds are illustrative examples of compounds of this aspect:

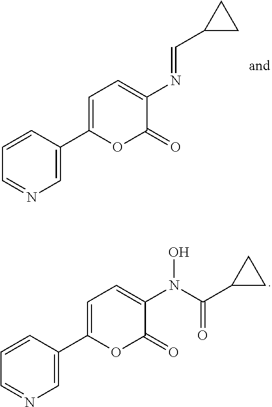 [SMILES: O=c1oc(-c2cccnc2)ccc1/N=C/C1CC1 ; O=C(C1CC1)N(O)c1ccc(-c2cccnc2)oc1=O]

In any of the above aspects and embodiments, heteroaryl groups may be any aromatic (i.e. a ring system containing 2(2n+1)π electrons) 5-10 membered ring system comprising from 1 to 4 heteroatoms independently selected from O, S and N (in other words from 1 to 4 of the atoms forming the ring system are selected from O, S and N). Thus, any heteroaryl groups may be independently selected from: 5 membered heteroaryl groups in which the heteroaromatic ring is substituted with 1-4 heteroatoms independently selected from O, S and N; and 6-membered heteroaryl groups in which the heteroaromatic ring is substituted with 1-3 (e.g.1-2) nitrogen atoms; 9-membered bicyclic heteroaryl groups in which the heteroaromatic system is substituted with 1-4 heteroatoms independently selected from O, S and N; 10-membered bicyclic heteroaryl groups in which the heteroaromatic system is substituted with 1-4 nitrogen atoms. Specifically, heteroaryl groups may be independently selected from: pyrrole, furan, thiophene, pyrazole, imidazole, oxazole, isoxazole, triazole, oxadiazole, thiadiazole, tetrazole; pyridine, pyridazine, pyrimidine, pyrazine, triazine, indole, isoindole, benzofuran, isobenzofuran, benzothiophene, indazole, benzimidazole, benzoxazole, benzthiazole, benzisoxazole, purine, quinoline, isoquinoline, cinnoline, quinazoline, quinoxaline, pteridine, phthalazine, naphthyridine. Heteroaryl groups may also be 6-membered heteroaryl groups in which the heteroaromatic ring is substituted with 1 heteroatomic group independently selected from O, S and NH and the ring also comprises a carbonyl group. Such groups include pyridones and pyranones. In any of the above aspects and embodiments, a heterocycloalkyl group is a 3-8 membered saturated or partially ring comprising 1 or 2 heteroatoms independently selected from O, S and N (in other words from 1 to 2 of the atoms forming the ring system are selected from O, S and N). By partially saturated it is meant that the ring may comprise one or two double bonds. This applies particularly to rings with from 5 to 8 members. The double bond will typically be between two carbon atoms but may be between a carbon atom and a nitrogen atom. Examples of heterocycloalkyl groups include; piperidine, piperazine, morpholine, thiomorpholine, pyrrolidine, tetrahydrofuran, tetrahydrothiophene, dihydrofuran, tetrahydropyran, dihydropyran, dioxane, azepine.

In any of the above aspects and embodiments, a haloalkyl group may have any amount of halogen substituents. The group may contain a single halogen substituent, it may have two or three halogen substituents, or it may be saturated with halogen substituents.

In an embodiment, in any R¹-R³³ group which contains an aryl or heteroaryl group, that aryl or heteroaryl group is optionally substituted, where chemically possible, by 1 to 4 substituents which are each independently selected at each occurrence from the group consisting of: R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a}; and OR^{a}; wherein R^{a} is selected from H, C₁-C₄ alkyl and C₁-C₄ haloalkyl; and wherein any two substituents on neighbouring atoms and comprising R^{a} groups may join up to form a ring.

In an embodiment, in any R¹-R³³ group which contains an alkyl, haloalkyl, cycloalkyl, or heterocycloalkyl group, that alkyl, haloalkyl, cycloalkyl or heterocycloalkyl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a}; and OR^{a}; wherein R^{a} is selected from H, C₁-C₄ alkyl and C₁-C₄ haloalkyl.

A group which is represented as SO₃R is typically a group having the form S(O)₂OR. A group which is represented as S(O)₂R is typically a group having the form S(O)₂R. A group which is represented as SO₂NR^{a}R^{a} is typically a group having the form S(O)₂NRR.

Compounds of the invention containing one or more asymmetric carbon atoms can exist as two or more stereoisomers. Where a compound of the invention contains a double bond such as a C═C or C═N group, geometric cis/trans (or Z/E) isomers are possible. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of the invention, including compounds exhibiting more than one type of isomerism, and mixtures of one or more thereof. Also included are acid addition or base salts wherein the counter ion is optically active, for example, d-lactate or l-lysine, or racemic, for example, dl-tartrate or dl-arginine.

The compounds of the invention may be obtained, stored and/or used in the form of an agronomically acceptable salt. Suitable salts include, but are not limited to, salts of acceptable inorganic acids such as hydrochloric, sulphuric, phosphoric, nitric, carbonic, boric, sulfamic, and hydrobromic acids, or salts of agronomically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids. The compounds may also be obtained, stored and/or used in the form of an N-oxide.

Cis/trans isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

The activity of the compounds of the present invention can be assessed by a variety of in silico, in vitro and in vivo assays. In silico analysis of a variety of compounds has been demonstrated to be predictive of ultimate in vitro and even in vivo activity.

The present invention also includes all environmentally acceptable isotopically-labelled compounds of formulae I to XX and their syntheses, wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Isotopically-labelled compounds can generally be prepared by conventional techniques known to those skilled in the art or by processes analogous to those described using an appropriate isotopically-labelled reagent in place of the non-labelled reagent previously employed.

Throughout this specification these abbreviations have the following meanings:

CDI—carbonyl diimidazole

DCE—dichloromethane

DCM—dichloromethane

DIAD—diisopropyl azodicarboxylate

DMAP—N,N-dimethyl-4-aminopyridine

DMF—dimethylformamide

DMSO—dimethylsulfoxide

EDCl—1-Ethyl-3-(3-dimethylaminopropyl)carbodiimide

HOAT—1-Hydroxy-7-azabenzotriazole

LDA—Lithium diisopropylamide

mCPBA—meta-chloroperbenzoic acid

pyr—pyridine

Selectfluor™—1-Chloromethyl-4-fluoro-1,4-diazoniabicyclo[2.2.2]octane bis(tetrafluoroborate)

Tf—trifluoromethylsulfonyl

THF—tetrahydrofuran

TBTU—O-(Benzotriazol-1-yl)-N,N,N′,N′-tetramethyluronium tetrafluoroborate

TMS—trimethylsilyl

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

If appropriate, the compounds of the invention can, at certain concentrations or application rates, be used as insecticides and/or acaricides.

According to another aspect of the present invention, there is provided a method for controlling insect and aracnid pests, the method comprising applying an agronomically effective and substantially non-phytotoxic (to the crop plant) quantity of a compound according to the invention to the seeds of the plants, to the plants themselves or to the area where it is intended that the plants will grow.

The pesticide may be applied as a seed treatment, foliar application, stem application, drench or drip application (chemigation) to the seed, the plant or to the fruit of the plant or to soil or to inert substrate (e.g. inorganic substrates like sand, rockwool, glasswool; expanded minerals like perlite, vermiculite, zeolite or expanded clay), Pumbe, Pyroclastic materials or stuff, synthetic organic substrates (e.g. polyurethane) organic substrates (e.g. peat, composts, tree waste products like coir, wood fibre or chips, tree bark) or to a liquid substrate (e.g. floating hydroponic systems, Nutrient Film Technique, Aeroponics).

In a further aspect, the present invention also relates to a insecticidal or acaricidal composition comprising an effective and non-phytotoxic amount of an active compound of the invention. The composition may further comprise one or more additional insecticides or acaricides.

The term “effective and non-phytotoxic amount” means an amount of pesticide according to the invention which is sufficient to control or destroy any of the targeted pests present or liable to appear in the crops and which does not have any significant detrimental effect on the crops or indeed has a positive effect on plant vigour and yield in the absence of target organism. The amount will vary depending on the pest to be controlled, the type of crop, the climatic conditions and the compounds included in the pesticidal composition. This amount can be determined by systematic field trials, which are within the capabilities of a person skilled in the art.

Depending on their particular physical and/or chemical properties, the active compounds of the invention can be formulated as solutions, emulsions, suspensions, powders, foams, pastes, granules, aerosols, microencapsulations in polymeric substances and in coating materials for seed, and also as ULV cold and warm fogging formulations.

The active compounds can be used neat, or in the form of a formulation, e.g. ready-to-use solutions, emulsions, water- or oil-based suspensions, powders, wettable powders, pastes, soluble powders, dusts, soluble granules, granules for broadcasting, suspoemulsion concentrates, natural substances impregnated with active compound, synthetic substances impregnated with active compound, fertilizers and also microencapsulations in polymeric substances. Application may be carried out, for example, by watering, spraying, atomizing, broadcasting, dusting, foaming, spreading, etc. It is also possible to apply the active compounds by the ultra-low volume method or to inject the preparation of active compound or the active compound itself into the soil. It is also possible to treat the seed of the plants.

Formulations containing the compounds of the invention are produced in a known manner, for example by mixing the compounds with extenders (e.g. liquid solvents and/or solid carriers), optionally with the use of surfactants (e.g. emulsifiers and/or dispersants and/or foam-formers). The formulations are prepared either in factories/production plants or alternatively before or during the application.

Auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to use, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide.

Suitable solid carriers are: for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE and/or -POP ethers, acid and/or POP-POE esters, alkylaryl and/or POP-POE ethers, fat- and/or POP-POE adducts, POE- and/or POP-polyol derivatives, POE- and/or POP-sorbitan- or -sugar adducts, alkyl or aryl sulphates, alkyl- or arylsulphonates and alkyl or aryl phosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

Further additives may be mineral and vegetable oils. It is also possible to add colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc. Other possible additives are perfumes, mineral or vegetable, optionally modified oils and waxes.

The formulations may also comprise stabilizers, e.g. low-temperature stabilizers, preservatives, antioxidants, light stabilizers or other agents which improve chemical and/or physical stability.

The formulations generally comprise between 0.01 and 98% by weight of active compound, preferably between 0.1 and 95% and particularly preferably between 0.5 and 90%.

The active compounds according to the invention can also be used as a mixture with other known insecticides and/or acaricides, for example, to improve the activity spectrum or to reduce or slow the development of resistance.

A mixture with other known active compounds such as nematicides, herbicides, fungicides, or bactericides, or with fertilizers and growth regulators, safeners or semiochemicals is also possible.

Exemplary application rates of the active compounds according to the invention are: when treating leaves: from 0.1 to 10 000 g/ha, preferably from 10 to 1000 g/ha, particularly preferably from 50 to 300 g/ha (when the application is carried out by watering or dripping, it is even possible to reduce the application rate, especially when inert substrates such as rock wool or perlite are used); when treating seed: from 2 to 200 g per 100 kg of seed, preferably from 2.5 to 150 g per 100 kg of seed, and particularly preferably from 2.5 to 25 g per 100 kg of seed, very particularly preferably from 2.5 to 12.5 g per 100 kg of seed; when treating the soil: from 0.1 to 10 000 g/ha, preferably from 1 to 5000 g/ha.

A formulation which could be used to administer the compounds, paricularly in the context of testing for activity, would be to supply all compounds as a 10% solution in DMSO. If there are solubility problems this can be helped by adding acetone (e.g. to dilute a DMSO solution/suspension by 50% resulting in a 5% solution of the compound in DMSO/acetone. The administration formulation is then obtained by adding the DMSO (or DMSO/acetone) solution to a 0.1% solution of Tween 20™ in water to give the required concentration. The result is likely to be an emulsion that can be sprayed. If crystallisation occurs, resulting in inconsistent results, further DMSO can be added to the test solution.

The compositions according to the invention are suitable for protecting any plant variety which is employed in agriculture, in the greenhouse, in forests or in horticulture and, in particular, cereals (e.g. wheat, barley, rye, millet and oats), maize, cotton, soya beans, rice, potatoes, sunflowers, beans, coffee, beet (for example sugar beet and fodder beet), peanuts, vegetables (e.g. tomatoes, cucumbers, onions and lettuce), lawns, fruit and nut trees (e.g. apples pears peaches nectarines, apricots, hazelnut, pecan, macadamia, pistachio), soft fruit (e.g. strawberries, raspberries, blackcurrants, redcurrants), grapevines, bananas, cocoa and ornamental plants.

The active compounds of the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, particularly insects and aracnids which are encountered in agriculture, in horticulture, in animal husbandry, in forests, in gardens and leisure facilities, in the protection of stored products and of materials, and in the hygiene sector. They may be preferably employed as crop protection agents.

Use as Insecticides/Acaricides

Some compounds of the invention may also have activity as insecticides/acaricides.

They may be active against normally sensitive and resistant species of pests and against all or some stages of development. The following are illustrative examples of pests that may be controlled by insecticidal/acaricidal compounds: from the order of the Anoplura (Phthiraptera), for example, Damalinia spp., Haematopinus spp., Linognathus spp., Pediculus spp., Trichodectes spp; from the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici; from the class of the Bivalva, for example, Dreissena spp; from the order of the Chilopoda, for example, Geophilus spp., Scutigera spp; from the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnostema consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp; from the order of the Collembola, for example, Onychiurus armatus; from the order of the Dermaptera, for example, Forficula auricularia; from the order of the Diplopoda, for example, Blaniulus guttulatus; from the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp; from the class of the Gastropoda, for example, Arion spp., Biomphalaria spp., Bulinus spp., Deroceras spp., Galba spp., Lymnaea spp., Oncomelania spp., Succinea spp; from the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

When used as insecticides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues. The active compound content of the use forms prepared from the commercially available formulations can vary within wide limits. The active compound concentration of the use forms can be from 0.00000001 to 95% by weight of active compound, preferably between 0.00001 and 1% by weight. The compounds are employed in a customary manner appropriate for the use forms.

The active compounds according to the invention may act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. The following are illustrative examples of parasites that may be controlled by insecticidal/acaricidal compounds: from the order of the Anoplurida, for example, Haematopinus spp., Linognathus spp., Pediculus spp., Phtirus spp., Solenopotes spp; from the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp; diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp; from the order of the Siphonapterida, for example, Pulex spp., Ctenocephalides spp., Xenopsylla spp., Ceratophyllus spp; from the order of the Heteropterida, for example, Cimex spp., Triatoma spp., Rhodnius spp., Panstrongylus spp; from the order of the Blattarida, for example, Blatta orientalis, Periplaneta americana, Blattela germanica, Supella spp; from the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp; from the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp. Each compound of the invention may have activity against one or more than one of the above organisms.

The active compounds according to the invention may also be suitable for controlling arthropods which infest agricultural productive livestock, such as, for example, cattle, sheep, goats, horses, pigs, donkeys, camels, buffalo, rabbits, chickens, turkeys, ducks, geese and bees, pets, such as, for example, dogs, cats, caged birds and aquarium fish, and also so-called test animals, such as, for example, hamsters, guinea pigs, rats and mice. By controlling these arthropods, cases of death and reductions in productivity (for meat, milk, wool, hides, eggs, honey etc.) should be diminished, so that more economic and easier animal husbandry is possible by use of the active compounds according to the invention.

The insecticidal/acaricidal compounds may be used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the insecticidal/acaricidal compounds can be used as formulations (for example powders, emulsions, free-flowing compositions), which comprise the active compounds in an amount of 1 to 80% by weight, directly or after 100- to 10 000-fold dilution, or they can be used as a chemical bath.

The insecticidal/acaricidal compounds of the invention may be used in the treatment of human disease, particularly parasitic infections e.g. those caused by mites, insects, helminths etc. Thus, the invention includes a method of treating a disease (e.g. a parasitic disease), the method comprising administering a therapeutic amount of an antifungal agent of the invention to a subject (e.g. a human subject) in need thereof. The compound may be formulated for topical administration to the infected area of the body or it may be formulated for oral or parenteral administration.

The insecticidal/acaricidal compounds may also have activity against insects which destroy industrial materials. The following are illustrative examples of pests that may be controlled by insecticidal/acaricidal compounds: Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus; Hymenopterons, such as Sirex juvencus, Urocerus gigas, Urocerus gigas taignus, Urocerus augur; Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus; Bristletails, such as Lepisma saccharina. Each compound of the invention may have activity against one or more than one of the above organisms.

Industrial materials in the present connection are to be understood as meaning non-living materials, such as, preferably, plastics, adhesives, sizes, papers and cardboards, leather, wood and processed wood products and coating compositions.

In domestic, hygiene and stored-product protection, the insecticidal/acaricidal compounds may also be suitable for controlling animal pests, in particular insects, arachnids and mites, which are found in enclosed spaces, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages.

The following are illustrative examples of pests that may be controlled by insecticidal/acaricidal compounds: from the order of the Scorpionidea, for example, Buthus occitanus; from the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae; from the order of the Araneae, for example, Aviculariidae, Araneidae; from the order of the Opiliones, for example, Pseudoscorpiones chelifer, Pseudoscorpiones cheiridium, Opiliones phalangium; from the order of the Isopoda, for example, Oniscus asellus, Porcellio scaber; from the order of the Diplopoda, for example, Blaniulus guttulatus, Polydesmus spp; from the order of the Chilopoda, for example, Geophilus spp; from the order of the Zygentoma, for example, Ctenolepisma spp., Lepisma saccharine, Lepismodes inquilinus; from the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa; from the order of the Saltatoria, for example, Acheta domesticus; from the order of the Dermaptera, for example, Forficula auricularia; from the order of the Isoptera, for example, Kalotermes spp., Reticulitermes spp; from the order of the Psocoptera, for example, Lepinatus spp., Liposcelis spp; from the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum; from the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa; from the order of the Lepidoptera, for example, Achroia grisella, Galleria mellonella, Plodia interpunctella, Tinea cloacella, Tinea pellionella, Tineola bisselliella; from the order of the Siphonaptera, for example, Ctenocephalides canis, Ctenocephalides felis, Pulex irritans, Tunga penetrans, Xenopsylla cheopis; from the order of the Hymenoptera, for example, Camponotus herculeanus, Lasius fuliginosus, Lasius niger, Lasius umbratus, Monomorium pharaonis, Paravespula spp., Tetramorium caespitum; from the order of the Anoplura, for example, Pediculus humanus capitis, Pediculus humanus corporis, Pemphigus spp., Phylloera vastatrix, Phthirus pubis; from the order of the Heteroptera, for example, Cimex hemipterus, Cimex lectularius, Rhodinus prolixus, Triatoma infestans. Each compound of the invention may have activity against one or more than one of the above organisms.

In the field of household insecticides, they may be used alone or in combination with other suitable active compounds, such as phosphoric esters, carbamates, pyrethroids, neonicotinoids, growth regulators or active compounds from other known classes of insecticides. They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

Detailed Description—Synthesis

The skilled man will appreciate that adaptation of methods known in the art could be applied in the manufacture of the compounds of the present invention.

For example, the skilled person will be immediately familiar with standard textbooks such as “Comprehensive Organic Transformations—A Guide to Functional Group Transformations”, RC Larock, Wiley-VCH (1999 or later editions); “March's Advanced Organic Chemistry—Reactions, Mechanisms and Structure”, M B Smith, J. March, Wiley, (5th edition or later); “Advanced Organic Chemistry, Part B, Reactions and Synthesis”, F A Carey, R J Sundberg, Kluwer Academic/Plenum Publications, (2001 or later editions); “Organic Synthesis—The Disconnection Approach”, S Warren (Wiley), (1982 or later editions); “Designing Organic Syntheses” S Warren (Wiley) (1983 or later editions); “Heterocyclic Chemistry”, J. Joule (Wiley 2010 edition or later); (“Guidebook To Organic Synthesis” R K Mackie and D M Smith (Longman) (1982 or later editions), etc., and the references therein as a guide.

The skilled person is familiar with a range of strategies for synthesising organic and particularly heterocyclic molecules and these represent common general knowledge as set out in text books such as Warren “Organic Synthesis: The Disconnection Approach”; Mackie and Smith “Guidebook to Organic Chemistry”; and Clayden, Greeves, Warren and Wothers “Organic Chemistry”.

The skilled chemist will exercise his judgement and skill as to the most efficient sequence of reactions for synthesis of a given target compound and will employ protecting groups as necessary. This will depend inter alia on factors such as the nature of other functional groups present in a particular substrate. Clearly, the type of chemistry involved will influence the choice of reagent that is used in the said synthetic steps, the need, and type, of protecting groups that are employed, and the sequence for accomplishing the protection/deprotection steps. These and other reaction parameters will be evident to the skilled person by reference to standard textbooks and to the examples provided herein.

Sensitive functional groups may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and references therein.

Certain compounds of the invention can be prepared using the methods described in the following general schemes.

Certain other compounds of the invention can be prepared according to or analogously to the methods described in Examples 1 to 4.

General Synthetic Schemes

Scheme A shows an illustrative route which can be used to prepare compounds of formulae XII and XIII.

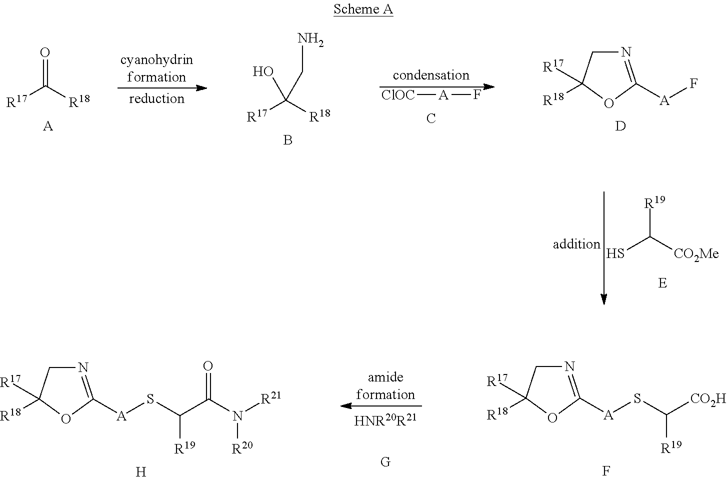

A typical synthesis according to Scheme A starts from a ketone A, which can be converted to hydroxy amine B via a cyanohydrin formation (e.g. using TMSCN optionally in DCM at room temperature followed by water at room temperature) and subsequent nitrile reduction (using e.g. LiAlH₄ in diethyl ether at room temperature). Reaction with acid chloride C (e.g. in the presence of SOCl₂ optionally in DCM) can provide oxazoline D. Thiol addition with thiol E (e.g. in the presence of K₂CO₃ optionally in DMF at room temperature) can, following ester hydrolysis (e.g. using NaOH optionally a 2 M solution with ethanol present as a cosolvent at room temperature), provide thiane F. Amide formation between acid F and amine G (e.g. using TBTU in the presence of Et₃N optionally in MeCN at room temperature) can provide compounds of formula H. The sulfide group can be oxidised to the sulfoxide or sulfone using 1.1 equivalents or 2.5. equivalents of mCPBA respectively (optionally in the presence of NaHCO₃ in DCM and water).

Scheme B shows an illustrative route which can be used to prepare compounds of formulae XII and XIV.

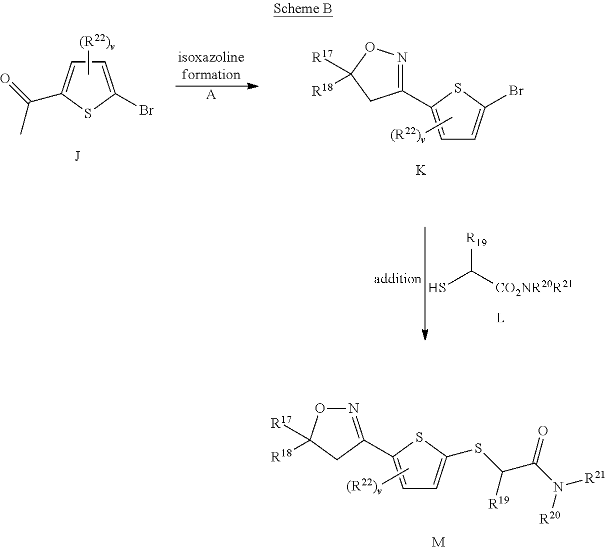

Reaction between ketones A and J (e.g. in two stages: in the presence of K₂CO₃, and triethylamine, optionally in DCE at 100° C.; and then treating the product with Bu₄NBr, 4M NaOH and hydroxylamine optionally in water at 0° C. can generate a isoxazoline K. Addition of thiol L (e.g. using NaH optionally in THF at room temperature) can provide compounds of formula M.

Scheme A shows an illustrative route which can be used to prepare compounds of formulae XII and XV.

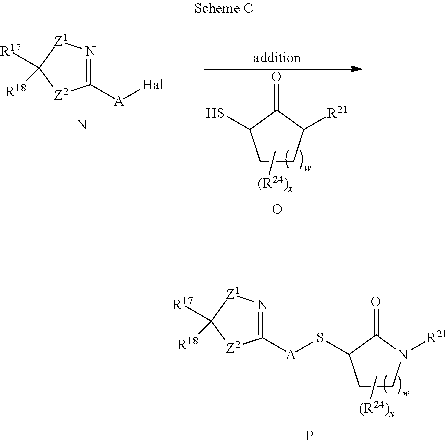

Addition of a thiol O to a halide N (specific examples of which are oxazolidine D and isoxazolidine K) can provide compounds of formula P. As described for Scheme A above, this product can be oxidised to the sulfoxide or sulfone.

Scheme D shows an illustrative route which can be used to prepare compounds of formulae XVI and XVII.

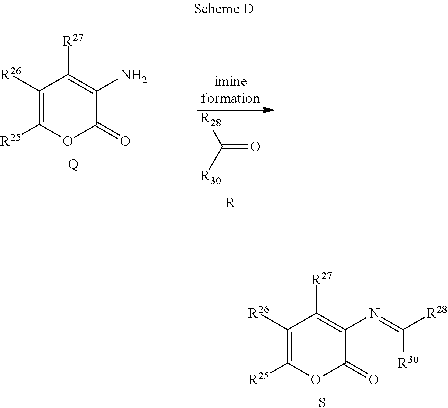

An imine forming reaction (e.g. using molecular sieves in an appropriate solvent, such as DCE, at room temperature) between amine Q and aldehyde or ketone R can provide imine S.

Scheme E shows an illustrative route which can be used to prepare compounds of formulae XVI, XVIII and XX.

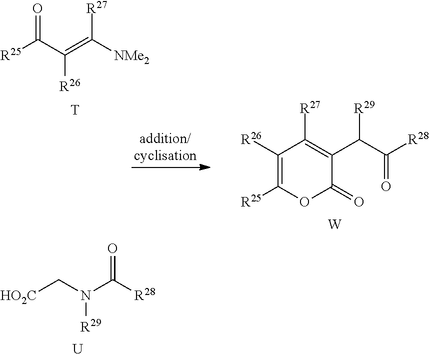

An addition cyclisation reaction (e.g. by heating in the presence of Ac2O and then treatin with a base, e.g. NaOH, at room temperature) between α,β-unsaturated ketone T and amido acid U can form a compound of formula W.

### EXAMPLES

Flash chromatography was carried out using silica gel (40-63 μm particles). Thin layer chromatography was carried out on pre-coated aluminium backed plates (Merck silica Keiselgel 60 F₂₅₄). Visualisation was carried out with UV light (254 nm) and by staining with either potassium permanganate, phosphomolybdic acid (PMA) or ninhydrin solutions. Where hexane is specified as a flash chromatography solvent, petroleum ether (b.p. 40-60° C.) can be used as an alternative.

All ¹H NMR spectra were obtained using either a Bruker Ultrashield 300 spectrometer or Bruker DPX300 spectrometer. Chemical shifts are expressed in parts per million (δ) and are referenced to the solvent. Coupling constants J are expressed in Hertz (Hz). ESI mass spectrometry was performed using a Bruker HCT Ultra LCMS instrument (Agilent 1200 Series LC with diode array detector and Bruker HCT Ultra Ion Trap MS) using a Phenomenex Luna 5u C18(2) 100 Å, 50×2.00 mm 5 micron LC column (solvent: 5-90% gradient of acetonitrile in water (with 1% formic acid). Flow rate 1.2 mL/min). El mass spectrometry was performed using a Varian Saturn 2100T GC/MS instrument with a FactorFour VF-5MS 30 m×0.25 mm capillary column. High resolution mass spectrometry (ESI) was performed using a Dionex UltiMate 3000 system.

All reagents were obtained from commercial suppliers and used as supplied unless otherwise stated.

### Example 1

### Butenolide Synthesis

### 3-[(6-Chloro-3-pyridyl)methyl-(2,2-difluoroethyl)amino]-2H-furan-5-one 1

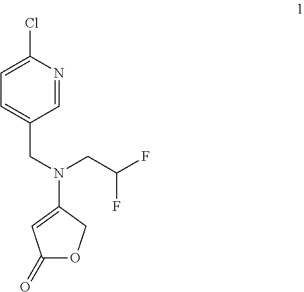 [SMILES: O=C1C=C(N(Cc2ccc(Cl)nc2)CC(F)F)CO1]

N-[(6-Chloro-3-pyridyl)methyl]-2,2-difluoro-ethanamine was prepared according to the procedure provided in WO 2008009360. N-[(6-Chloro-3-pyridyl)methyl]-2,2-difluoro-ethanamine (347 mg, 1.68mmol), tetronic acid (236 mg, 2.38 mmol) and p-toluenesulfonic acid (16 mg, 0.08 mmol) were added to toluene and stirred at reflux for 6 h, after which time TLC showed the complete consumption of the starting material. The solvent was removed in vacuo, and the residue was purified by column chromatography, eluting with 3% MeOH in DCM to afford the product (200 mg, 41%) as a colourless oil.

¹H NMR δ_{H} (CDCl₃, 300 MHz): 8.30 (s, 1H), 7.54 (dd, J=8.1, 2.7 Hz, 1H), 7.40 (d, J=8.1 Hz, 1H), 5.96 (tt, J=54.6, 3.3 Hz, 1H), 4.88 (s, 1H), 4.83 (s, 2H), 4.52 (s, 2H), 3.52 (dt, J=14.7, 3.3 Hz, 1H)

ESI-MS 599.4 [2M+Na]⁺

Compound 1 (Flupyradifurone) does not form part of the invention and is included for comparative purposes only.

### Ethyl 2-[(6-chloro-3-pyridyl)methylamino]acetate 2

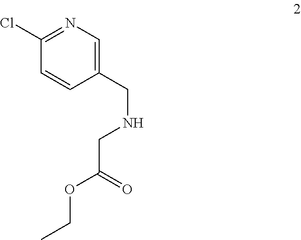

To a solution of of 2-chloro-5-chloromethyl pyridine (500 mg, 3.1 mmol) in acetonitrile (50 mL) was added triethylamine (2.2 mL, 9.3 mmol) and ethyl glycinate hydrochloride (864 mg, 6.2 mmol) and the mixture heated to 50° C. for 16 h. The solvent was removed in vacuo and the crude material was purified by flash chromatography on silica (solvent 5% MeOH/DCM) to afford the title compound as a yellow oil (300 mg, 43%).

¹H NMR δ_{H} (CDCl₃, 300 MHz): 8.34 (d, J=2.4 Hz, 1H), 7.70 (dd, J=8.4, 2.4 Hz, 1H), 7.30 (d, J=8.4 Hz, 1H), 4.20 (q, J=6.9 Hz, 2H), 3.82 (s, 2H), 3.40 (s, 2H), 1.29 (t, J=7 Hz, 3H).

### Ethyl 2-[(6-chloro-3-pyridyl)methyl-(5-oxo-2H-furan-3-yl)amino]acetate 3

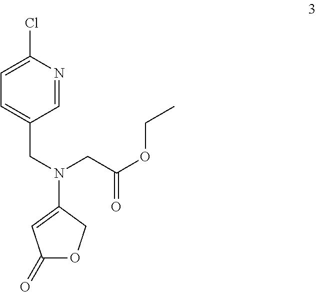 [SMILES: CCOC(=O)CN(Cc1ccc(Cl)nc1)C1=CC(=O)OC1]

Ethyl 2-[(6-chloro-3-pyridyl)methylamino]acetate 2 (300 mg, 1.3 mmol), tetronic acid (184 mg, 1.8 mmol) and p-toluenesulfonic acid (13 mg, 0.07 mmol) were added to toluene and stirred at reflux for 5 h, after which time TLC showed the complete consumption of starting material. The solvent was removed in vacuo, and the residue was purified by column chromatography, eluting with 3% MeOH in DCM to afford the product (197 mg, 48%) as a colourless oil.

¹H NMR δ_{H} (CDCl₃, 300 MHz): 8.30 (d, J=2.4 Hz, 1H), 7.61 (dd, J=8.1, 2.7 Hz, 1H), 7.37 (d, J=8.1 Hz, 1H), 5.31 (s, 1H), 4.80 (d, J=9 Hz, 2H), 4.46 (s, 2H), 4.22 (dd, J=14.4, 7.2 Hz, 2H), 3.83 (s, 2H), 1.28 (t, J=7.2 Hz, 3H)

ESI-MS 311.1 [M+H]⁺

### N-[(6-Chloro-3-pyridyl)methyl]-2,2-dimethoxy-ethanamine 4

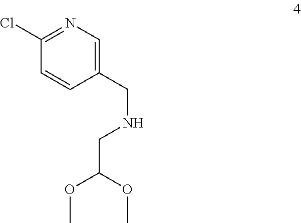

To a solution of 2-chloro-5-chloromethyl pyridine (500 mg, 3 mmol) in acetonitrile (20mL) was added triethylamine (848 μL, 6 mmol) and amino acetaldehyde dimethyl acetal (1.7 mL, 15 mmol) and the mixture heated to 50° C. for 24 h. The solvent was removed in vacuo and the crude material was purified by flash chromatography on silica (solvent 2% MeOH/DCM) to afford the title compound as a yellow oil (600 mg, 85%).

¹H NMR δ_{H} (CDCl₃, 300 MHz) 8.26 (d, J=2.5 Hz, 1H), 7.59 (dd, J=8.2, 2.5 Hz, 1H), 7.21 (d, J=8.2 Hz, 1H), 4.39 (t, J=6.0 Hz, 1H), 3.73 (s, 2H), 3.32 (s, 6H), 2.65 (d, J=6.0 Hz, 2H). El-MS 231.1 [M+H]⁺

### 3-[(6-Chloro-3-pyridyl)methyl-(2,2-dimethoxyethyl)amino]-2H-furan-5-one 5

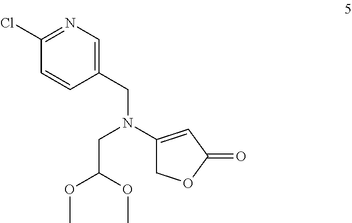 [SMILES: COC(CN(Cc1ccc(Cl)nc1)C1=CC(=O)OC1)OC]

To a solution of 3 N-[(6-Chloro-3-pyridyl)methyl]-2,2-dimethoxy-ethanamine 4 (540 mg, 2.3 mmol) in toluene (7 mL) was added 3-hydroxy-2H-furan-5-one (329 mg, 3.3 mmol) and p-toluenesulfonic acid 22 mg, 0.12 mmol) and the mixture heated to reflux for 6 h (with vigorous stirring) after which time the solvent was removed in vacuo and the crude material was purified by flash chromatography on silica gel (solvent 1% MeOH/DCM moving to 3% MeOH/DCM) to afford the title compound as a purple oil (130 mg, 29%).

¹H NMR δ_{H} (CDCl₃, 300 MHz) 8.28 (d, J=2.5 Hz, 1H), 7.54 (dd, J=8.2, 2.5 Hz, 1H), 7.37 (d, J=8.2 Hz, 1H), 4.84 (br s, 2H), 4.74 (br s, 1H), 4.50 (br s, 2H), 4.43 (br s, 1H), 3.42 (s, 6H), 3.26 (d, J=4.8 Hz, 2H). ESI-MS 313.1 [M+H]⁺

### 1-[(4-Chlorophenyl)methyl]-6H-furo[3,4-b]pyrrol-4-one 6

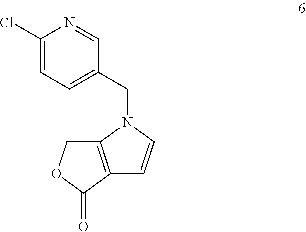 [SMILES: O=C1OCc2c1ccn2Cc1ccc(Cl)nc1]

N-[(6-chloro-3-pyridyl)methyl]-2,2-dimethoxy-ethanamine 4 (540 mg, 2.3 mmol), tetronic acid (327 mg, 3.3 mmol) and p-toluenesulfonic acid (22 mg, 0.11 mmol) were added to toluene and stirred at reflux for 5 h, after which time TLC showed the complete consumption of starting material. The solvent was removed in vacuo and the residue was purified by column chromatography, eluting with 3% MeOH in DCM to afford the product (70 mg, 12%) as a light brown solid.

¹H NMR δ_{H} (CDCl₃, 300 MHz): 8.34 (s, 1H), 7.47 (d, J=9 Hz, 1H), 7.40 (d, J=9 Hz, 1H), 6.84 (d, J=3 Hz, 1H), 6.49 (d, J=3 Hz, 1H), 5.09 (s, 2H), 4.84 (s, 2H).

ESI-MS 519.6 [2M+Na]⁺

### 2-[(6-Chloro-3-pyridyl)methyl-(5-oxo-2H-furan-3-yl)amino]acetonitrile 7

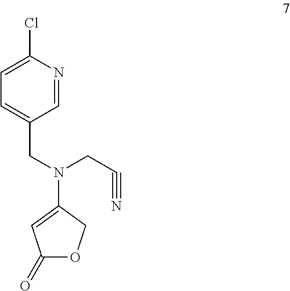 [SMILES: N#CCN(Cc1ccc(Cl)nc1)C1=CC(=O)OC1]

2-[(6-Chloro-3-pyridyl)methylamino]acetonitrile was prepared according to the procedure provided in JP 05163241. 2-[(6-Chloro-3-pyridyl)methylamino]acetonitrile (146 mg, 0.8 mmol)), 3-hydroxy-2H-furan-5-one (113 mg, 1.3 mmol) and p-toluenesulfonic acid (8 mg, 0.04 mmol) were added to toluene, and stirred at reflux for 2 h, after which time TLC showed the complete consumption of the pyridine starting material. The solvent was removed in vacuo and the residue was purified by flash chromatography on silica gel (solvent 3% MeOH/1% triethylamine/DCM) to afford the title compound as a light brown solid (52 mg, 25%).

¹H NMR δ_{H} (CDCl₃, 300 MHz): 8.14 (d, J=2.1 Hz, 1H), 7.45 (dd, J=8.4, 2.4 Hz, 1H), 7.17 (d, J=8.1 Hz, 1H), 4.78 (s, 1H), 4.70 (s, 2H), 4.30 (s, 2H), 4.05 (s, 2H). ESI-MS 264.3 [M+H]⁺

### Example 2

### Pyridine Synthesis

### 5-(1-methylsulfinylethyl)-2-(trifluoromethyl)pyridine 8

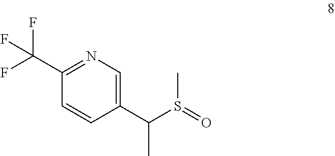

5-(1-Methylsulfanylethyl)-2-(traluoromethyl)pyridine was prepared according to the procedure provided in WO 2010/002577. To a mixture of 5-(1-methylsulfanylethyl)-2-(trifluoromethyl)pyridine (500 mg, 2.3 mmol) dissolved in acetonitrile (3 mL) and cooled to 0° C. was added iodobenzene diacetate (729 mg, 2.3 mmol) in one portion. The solution was stirred at this temperature for 10 minutes before the ice bath was removed and the solution allowed to stir at room temperature for 40 h. An aqueous solution of sodium metabisulfite (3 mL) was added and the mixture extracted with hexane (2×20 mL). The remaining aqueous solution was then extracted with ethyl acetate (3×20 mL) with the organic fractions dried over MgSO₄ and the solvent removed in vacuo. The resulting residue was purified by column chromatography, eluting with 3% MeOH in DCM to afford the product (280 mg, 52%) as a white solid (mixture of diastereoisomers).

¹H NMR δ_{H} (CDCl₃, 300 MHz): 8.67 and 8.62 (2 br d, J=1.8 Hz, 1H), 7.92 and 7.84 (2 br dd, J=8.2, 1.8 Hz, 1 H), 7.76 and 7.73 (2 br d, J=8.2 Hz, 1H), 3.96 and 3.80 (2 q, J=7.2 Hz, 1H), 1.80 and 1.76 (2 d, J=7.2 Hz, 3H). ESI-MS 260.0 [M+Na]⁺

### [Methyl-[1-[6-(trifluoromethyl)-3-pyridyl]ethyl]-λ⁴-sulfanylidene]cyanamide 9

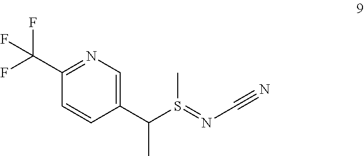 [SMILES: CC(c1ccc(C(F)(F)F)nc1)/S(C)=N/C#N]

To a mixture of 5-(1-methylsulfanylethyl)-2-(trifluoromethyl)pyridine 8 (500 mg, 2.3 mmol) and cyanamide (95 mg, 2.3 mmol) dissolved in acetonitrile (3 mL) and cooled to 0° C. was added iodobenzene diacetate (729 mg, 2.3 mmol) in one portion. The solution was stirred at this temperature for 10 minutes before the ice bath was removed and the solution allowed to stir at room temperature for 16 h. An aqueous solution of sodium metabisulfite (3 mL) was added and the mixture extracted with hexane (2×20 mL). The remaining aqueous solution was then extracted with ethyl acetate (3×20 mL) with the organic fractions dried over MgSO₄ and the solvent removed in vacuo. The resulting residue was purified by column chromatography, eluting with 3% MeOH in DCM to afford the product (469 mg, 79%) as a yellow oil (mixture of diastereoisomers).

¹H NMR δ_{H} (CDCl₃, 300 MHz): 8.66 and 8.62 (2 br dd, J=1.8 Hz, 1H), 7.96 and 7.85 (2 br dd, J=8.2, 1.8 Hz, 1H), 7.74 and 7.73 (2 br d, J=8.2 Hz, 1H), 4.35 and 4.29 (2 q, J=7.1 Hz, 1H), 2.57 and 2.56 (2 s, 3H), 1.85 and 1.83 (2 d, J=7.1 Hz, 3H). ESI-MS 545.3 [2M+Na]⁺

### N-[Methyl-[1-[6-(trifluoromethyl)-3-pyridyl]ethyl]-λ⁴-sulfanylidene]methanesulfonamide 10

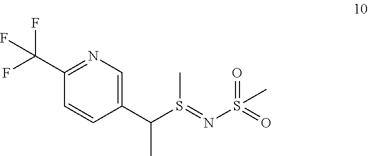 [SMILES: CC(c1ccc(C(F)(F)F)nc1)/S(C)=N/S(C)(=O)=O]

To a mixture of 5-(1-methylsulfanylethyl)-2-(trifluoromethyl)pyridine 8 (500 mg, 2.3 mmol) and methanesulfonamide (215 mg, 2.3 mmol) dissolved in acetonitrile (3 mL) and cooled to 0° C. was added iodobenzene diacetate (729 mg, 2.3 mmol) in one portion. The solution was stirred at this temperature for 10 minutes before the ice bath was removed and the solution allowed to stir at room temperature for 40 h. An aqueous solution of sodium metabisulfite (3 mL) was added and the mixture extracted with hexane (2×20 mL). The remaining aqueous solution was then extracted with ethyl acetate (3×20 mL) with the organic fractions dried over MgSO₄ and the solvent removed in vacuo. The resulting residue was purified by column chromatography, eluting with 3% MeOH in DCM to afford the product (440 mg, 62%) as a white solid (mixture of diastereoisomers).

¹H NMR δ_{H} (CDCl₃, 300 MHz): 8.74 and 8.67 (2 br d, J=1.8 Hz, 1H), 8.09 and 7.99 (2 br dd, J=8.2, 1.8 Hz, 1H), 7.82 and 7.81 (2 br d, J=8.2 Hz, 1H), 4.43 and 4.24 (2 q, J=7.4 Hz, 1H), 2.96 and 2.86 (2 s, 3H), 2.56 and 2.50 (2 s, 3H), 1.89 and 1.88 (2 d, J=7.4 Hz, 3H). ESI-MS 651.2 [2M+Na]⁺

### N-[Methyl-oxo-[1-[6-(trifluoromethyl)-3-pyridyl]ethyl]-λ⁶-sulfanylidene]cyanamide 11

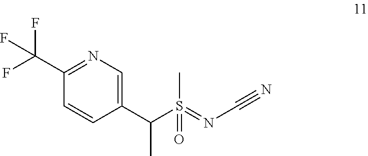 [SMILES: CC(c1ccc(C(F)(F)F)nc1)S(C)(=O)=NC#N]

To a solution of 5-[1-(methylsulfonimidoyl)ethyl]-2-(trifluoromethyl)pyridine (500 mg, 2.0 mmol) dissolved in dichloromethane (5 mL) was added DMAP (242 mg, 2.0 mmol) followed by cyanogen bromide (252 mg, 2.3 mmol) and the solution stirred at room temperature for 16 h. The reaction mixture was diluted with dichloromethane (10 mL) and washed with water (10 mL) before being dried over MgSO₄ and the solvent removed in vacuo. The resulting residue was purified by column chromatography, eluting with 2% MeOH in DCM to afford the product (386 mg, 70%) as a pale yellow oil (mixture of diastereoisomers).

¹H NMR δ_{H} (CDCl₃, 300 MHz): 8.81 and 8.80 (br d, J=2.0 Hz, 1H), 8.12 (br dd, J=8.2, 1.7 Hz, 1H), 7.84 (br d, J=8.2 Hz, 1H), 4.73 (q, J=7.1 Hz, 1H), 3.16 and 3.12 (2 s, 3H), 2.02 (d, J=7.1 Hz, 3H). ESI-MS 300.0 [M+Na]⁺

Compound 11 (sulfoxaflor) does not form part of the invention and is included for comparative purposes only.

### N-[Methyl-oxo-[1-[6-(trifluoromethyl)-3-pyridyl]ethyl]-λ⁶-sulfanylidene]acetamide 12

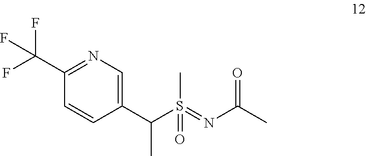 [SMILES: CC(=O)N=S(C)(=O)C(C)c1ccc(C(F)(F)F)nc1]

To a solution of 5-[1-(methylsulfonimidoyl)ethyl]-2-(trifluoromethyl)pyridine (1 g, 4.0 mmol) dissolved in dichloromethane(10 mL) cooled to 0° C. was added triethylamine (0.52 g, 719 μL, 5.2 mmol) followed by acetyl chloride (0.37 g, 340 μL, 4.8 mmol). The reaction was allowed to warm to room temperature at which point the solution was stirred for 16 h. The reaction mixture was diluted with dichloromethane (20 mL) and washed with water (20 mL) before being dried over MgSO₄ and the solvent removed in vacuo. The resulting residue was purified by column chromatography, eluting with DCM moving to 2% MeOH in DCM to afford the product (1.06 g, 91%) as a brown oil (mixture of diastereoisomers).

¹H NMR δ_{H} (CDCl₃, 300 MHz): 8.81 and 8.76 (2 br d, J=2.0 Hz, 1H), 8.09 and 8.03 (2 br dd, J=8.2, 2.0 Hz, 1H), 7.79 and 7.78 (2 br d, J=8.2 Hz, 1H), 5.04 and 5.01 (2 q, J=7.2 Hz, 1H), 3.25 and 3.03 (2 s, 3H), 2.16 and 2.05 (2 s, 3H) 1.92 and 1.84 (2 d, J=7.2 Hz, 3H). ESI-MS 611.3 [2M+Na]⁺

### Methyl N-[methyl-oxo-[1-[6-(trifluoromethyl)-3-pyridyl]ethyl]-λ⁶-sulfanylidene]carbamate 13

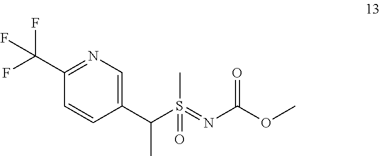 [SMILES: COC(=O)N=S(C)(=O)C(C)c1ccc(C(F)(F)F)nc1]

To a solution of 5-[1-(methylsulfonimidoyl)ethyl]-2-(trifluoromethyl)pyridine (500 mg, 2.0 mmol) dissolved in dichloromethane (12 mL) was added DMAP (249 mg, 2.0 mmol) followed by methyl chloroformate (374 mg, 308 μL, 4.0 mmol) and the solution stirred at room temperature for 3 h. The reaction mixture was diluted with dichloromethane (20 mL) and washed with 1N HCl (20 mL) before being dried over MgSO₄ and the solvent removed in vacuo. The resulting residue was purified by column chromatography, eluting with 2% MeOH in DCM to afford the product (440 mg, 72%) as a yellow viscous oil (mixture of diastereoisomers).

¹H NMR δ_{H} (CDCl₃, 300 MHz): 8.71 (br d, J=1.6 Hz, 1H), 8.04-8.01 (m, 1H), 7.71 and 7.69 (2 br d, J=8.2 Hz, 1H), 4.77 and 4.78 (2 q, J=7.1 Hz, 1H), 3.66 and 3.62 (2 s, 3H), 3.06 and 3.00 (2 s, 3H), 1.84 and 1.86 (2 d, J=7.1 Hz, 3H). ESI-MS 643.3 [2M+Na]⁺

### N-[methyl-oxo-[1-[6-(trifluoromethyl)-3-pyridyl]ethyl]-λ⁶-sulfanylidene]methane-sulfonamide 14

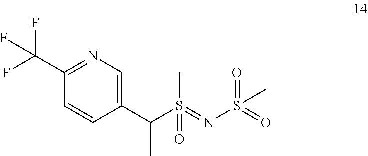 [SMILES: CC(c1ccc(C(F)(F)F)nc1)S(C)(=O)=NS(C)(=O)=O]

To a solution of 5-[1-(methylsulfonimidoyl)ethyl]-2-(trifluoromethyl)pyridine (400 mg, 1.6 mmol) and triethylamine (193 mg, 265 μL, 1.9 mmol) dissolved in dichloromethane (8 mL) cooled to 0° C. was added methanesulfonyl chloride (218 mg, 147 μL, 4.8 mmol). The reaction was allowed to warm to room temperature at which point the solution was stirred for 16 h. The reaction mixture was diluted with dichloromethane (20 mL) and washed with water (20 mL) before being dried over MgSO₄ and the solvent removed in vacuo. The resulting residue was purified by column chromatography, eluting with DCM moving to 2% MeOH in DCM to afford the product (450 mg, 86%) as a clear viscous oil (mixture of diastereoisomers).

¹H NMR δ_{H} (CDCl₃, 300 MHz): 8.83 and 8.81 (2 br d, J=1.8 Hz, 1H), 8.14 and 8.11 (2 br dd, J=8.2, 1.8 Hz, 1H), 7.81 and 7.79 (2 br d, J=8.2 Hz, 1H), 4.81 and 4.76 (2 q, J=7.1 Hz, 1H), 3.21 and 3.20 (2 s, 3H), 3.11 and 3.08 (2 s, 3H) 1.99 and 1.97 (2 d, J=7.1 Hz, 3H). ESI-MS 683.3 [2M+Na]⁺

### Example 3

### Oxazoline and Isoxazoline Synthesis

### 2-[4-[5-(3,5-dichlorophenyl)-5-(trifluoromethyl)-4H-isoxazol-3-yl]-2-methyl-phenyl]sulfanyl-N-(2,2,2-trifluoroethyl)acetamide 15

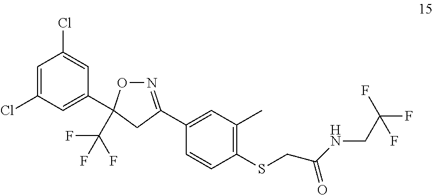 [SMILES: Cc1cc(C2=NOC(c3cc(Cl)cc(Cl)c3)(C(F)(F)F)C2)ccc1SCC(=O)NCC(F)(F)F]

5-(3,5-Dichlorophenyl)-3-(4-fluoro-3-methyl-phenyl)-5-(trifluoromethyl)-4H-isoxazole was prepared according to the procedure provided in WO 2012/156400. 2-Sulfanyl-N-(2,2,2-trifluoroethyl)acetamide was prepared according to the procedure provided in WO 2012/156400. 5-(3,5-dichlorophenyl)-3-(4-fluoro-3-methyl-phenyl)-5-(trifluoromethyl)-4H-isoxazole (445 mg, 1.13 mmol), 2-sulfanyl-N-(2,2,2-trifluoroethyl)acetamide (295 mg, 1.70 mmol) and potassium carbonate (235 mg, 1.70 mmol) were mixed in DMF (5 mL) and the solution heated to 100° C. After 16 h and 40 h, further portions of 2-sulfanyl-N-(2,2,2-trifluoroethyl)acetamide (295 mg, 1.70 mmol) and potassium carbonate (235 mg, 1.70 mmol) were added. After 48 h, the reaction was diluted with EtOAc (5 mL) and washed with H₂O (3×5 mL). The organic fraction was dried over MgSO₄ and the solvent remove in vacuo to afford a crude product that was purified by flash chromatography on silica gel (solvent 10% EtOAc/petrol moving to 20% EtOAc/petrol) to afford the title compound (210 mg, 34%).

¹H NMR OH (300 MHz, CDCl₃): 7.55-7.52 (m, 3H), 7.45-7.43 (m, 2H), 7.11 (d, J=9.0 Hz, 1H), 6.96 (br t, J=6.6 Hz, 1H), 4.07 (d, J=17.2 Hz, 1H), 3.92 (dq, J=9.0 and 6.6 Hz, 2H), 3.77 (s, 2H), 3.68 (d, J=17.2 Hz, 1H), 2.41 (s, 3H). ESI-MS 545.22 [MH⁺].

Compound 15 forms part of the prior art (WO2012/156400) and is included for reference purposes only.

### N-[2-(3,5-Dichlorophenyl)-3,3,3-trifluoro-2-hydroxy-propyl]-4-fluoro-3-methyl-benzamide 16

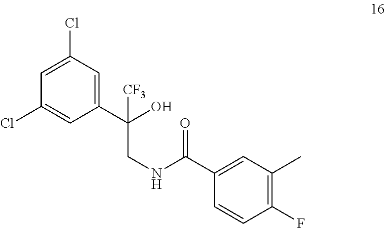 [SMILES: Cc1cc(C(=O)NCC(O)(c2cc(Cl)cc(Cl)c2)C(F)(F)F)ccc1F]

α-(Aminomethyl)-3,5-dichloro-α-(trifluoromethyl)-benzenemethanol was prepared according to the procedure provided in WO 2011/051455. To a solution of 4-fluoro-3-methylbenzoic acid (118 mg, 0.77 mmol) in DMF (3 ml) was added HOBt (104 mg, 0.77 mmol) and the mixture stirred for 15 minutes at room temperature before EDCl (147 mg, 0.77 mmol) was added and the mixture stirred for a further 15 minutes. α-(Aminomethyl)-3,5-dichloro-α-(trifluoromethyl)-benzenemethanol (210 mg, 0.77 mmol) and triethylamine (129 μl, 0.92 mmol) in DMF (3 ml) were added and the mixture stirred at room temperature for 16 h. The reaction mixture was diluted with EtOAc (10 ml) and water (15 ml) and the organic layer separated. The aqueous layer was further extracted with EtOAc (2×10 ml) and the combined organic phases washed with brine (10 ml) and dried over MgSO₄. The solvents were removed in vacuo and the residue purified by flash chromatography on silica gel (solvent 10% EtOAc/hexane) to give the title compound (320 mg, quantitative yield).

¹H NMR OH (300 MHz, CDCl₃): 7.64 (m, 1H), 7.56 (d, J=3.0 Hz, 2H), 7.55-7.50 (m, 1H), 7.36 (t, J=3.0 Hz, 1H), 7.04 (t, J=7.5 Hz, 1H), 6.94 (br t, J=6.0 Hz, 1H), 4.27 (dd, J=15.0 and 6.0 Hz, 1H), 3.94 (dd, J=15.0 and 6.0 Hz, 1H), 2.30 (s, 3H); ESI-MS 410.0 [MH⁺].

### 5-(3,5-Dichlorophenyl)-2-(4-fluoro-3-methyl-phenyl)-5-(trifluoromethyl)-4H-oxazole 17

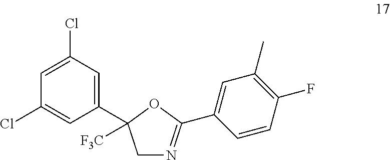 [SMILES: Cc1cc(C2=NCC(c3cc(Cl)cc(Cl)c3)(C(F)(F)F)O2)ccc1F]

N-[2-(3,5-Dichlorophenyl)-3,3,3-trifluoro-2-hydroxy-propyl]-4-fluoro-3-methyl-benzamide 16 (320 mg, 0.78 mmol), triphenylphosphine (257 mg, 0.98 mmol) and DIAD (192 μl, 0.98 mmol) were combined in THF (6 ml) and stirred at room temperature for 48 h. The solvent was removed in vacuo and the residue purified by flash chromatography on silica gel (10% EtOAc in petrol ethers) to give the title compound (130 mg, 42%).

¹H NMR δ_{H} (300 MHz, CDCl3): 7.95-7.84 (m, 2H), 7.46-7.43 (m, 3H), 7.13 (t, J=9.0 Hz, 1H), 4.76 (d, J=15.0 Hz, 1H), 4.29 (d, J=15.0 Hz, 1H), 2.37 (s, 3 H); ESI-MS 392.0 [MH⁺].

### 2-[4-[5-(3,5-dichlorophenyl)-5-(trifluoromethyl)-4H-oxazol-2-yl]-2-methyl-phenyl]sulfanyl-N-(2,2,2-trifluoroethyl)acetamide 18

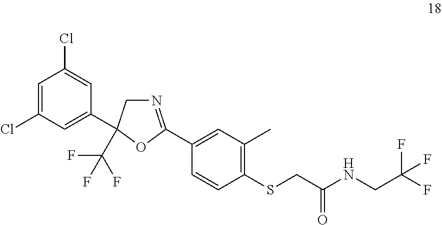 [SMILES: Cc1cc(C2=NCC(c3cc(Cl)cc(Cl)c3)(C(F)(F)F)O2)ccc1SCC(=O)NCC(F)(F)F]

5-(3,5-dichlorophenyl)-2-(4-fluoro-3-methyl-phenyl)-5-(trifluoromethyl)-4H-oxazole 17 (130 mg, 0.33 mmol), 2-sulfanyl-N-(2,2,2-trifluoroethyl)acetamide (86 mg, 0.50 mmol) and potassium carbonate (69 mg, 0.50 mmol) were mixed in DMF (2 mL) and the solution heated to 80° C. After 8 h and 24 h, further portions of 2-sulfanyl-N-(2,2,2-trifluoroethyl)acetamide (86 mg, 0.50 mmol) and potassium carbonate (69 mg, 0.50 mmol) were added. After 48 h, the reaction was diluted with EtOAc (5 mL) and washed with H₂O (3×5 mL). The organic fraction was dried over MgSO₄ and the solvent remove in vacuo to afford a crude product that was purified by flash chromatography on silica gel (solvent 10% EtOAc/petrol moving to 30% EtOAc/petrol) to afford the title compound (75 mg, 41%).

¹H NMR δ_{H} (300 MHz, CDCl₃): 7.84 (m, 2H), 7.46-7.41 (m, 3H), 7.16 (d, J=7.2 Hz, 1H), 6.98 (br m, 1H), 4.76 (d, J=15.4 Hz, 1H), 4.30 (d, J=15.0 Hz, 1H), 3.93 (dq, J=9.0 and 6.0 Hz, 2H), 3.76 (s, 2H), 2.46 (s, 3H). ESI-MS 545.2 [MH⁺].

### 2-[[5-[5-(3,5-dichlorophenyl)-5-(trifluoromethyl)-4H-isoxazol-3-yl]-2-thienyl]sulfanyl]-N-(2,2,2-trifluoroethyl)acetamide 19

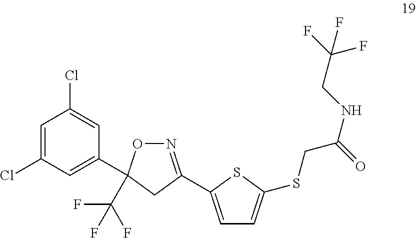 [SMILES: O=C(CSc1ccc(C2=NOC(c3cc(Cl)cc(Cl)c3)(C(F)(F)F)C2)s1)NCC(F)(F)F]

3-(5-Bromo-2-thienyl)-5-(3,5-dichlorophenyl)-5-(trifluoromethyl)-4H-isoxazole was prepared according to the procedure provided in WO 2010/070068. 3-(5-Bromo-2-thienyl)-5-(3,5-dichlorophenyl)-5-(trifluoromethyl)-4H-isoxazole (200 mg, 0.45 mmol), 2-sulfanyl-N-(2,2,2-trifluoroethyl)acetamide (78 mg, 0.45 mmol) and potassium carbonate (93 mg, 0.67 mmol) were mixed in DMF (2 mL) and the solution heated to 100° C. After 16 h, a further portion of 2-sulfanyl-N-(2,2,2-trifluoroethyl)acetamide (78 mg, 0.50 mmol) was added. After 20 h, the reaction was diluted with EtOAc (5 mL) and washed with H₂O (3×5 mL). The organic fraction was dried over MgSO₄ and the solvent removed in vacuo to afford a crude product that was purified by flash chromatography on silica gel (solvent 40% EtOAc/petrol) to afford the title compound (106 mg, 44%).

¹H NMR OH (CDCl₃, 300 MHz): 7.50-7.49 (m, 2H), 7.45 (t, J=1.8 Hz, 1H), 7.11 (s, 2H), 6.75 (br t, J=6.6 Hz, 1H), 4.05 (d, J=17.0 Hz, 1H), 3.97 (dq, J=6.6 and 9.0 Hz, 2H), 3.66 (d, J=17.0 Hz, 1H), 3.63 (s, 2H). ESI-MS 537.1[MH]⁺.

### 2-[[5-[5-(3,5-dichlorophenyl)-5-(trifluoromethyl)-4H-isoxazol-3-yl]-3-methyl-2-thienyl]sulfanyl]-N-(2,2,2-trifluoroethyl)acetamide 20

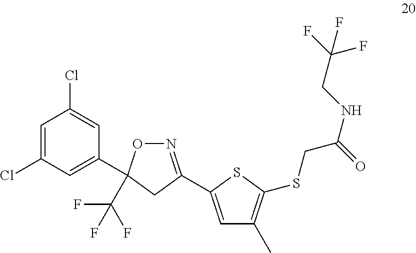 [SMILES: Cc1cc(C2=NOC(c3cc(Cl)cc(Cl)c3)(C(F)(F)F)C2)sc1SCC(=O)NCC(F)(F)F]

3-(5-Bromo-4-methyl-2-thienyl)-5-(3,5-dichlorophenyl)-5-(trifluoromethyl)-4H-isoxazole was prepared according to the procedure provided in WO 2011/157748. 3-(5-Bromo-4-methyl-2-thienyl)-5-(3,5-dichlorophenyl)-5-(trifluoromethyl)-4H-isoxazole (520 mg, 1.13 mmol), 2-sulfanyl-N-(2,2,2-trifluoroethyl)acetamide (294 mg, 1.70 mmol) and potassium carbonate (235 mg, 1.70 mmol) were mixed in DMF (2 mL) and the solution heated to 100° C. After 4 h, the reaction was diluted with EtOAc (20 mL) and washed with H₂O (3×10 mL). The organic fraction was dried over MgSO₄ and the solvent removed in vacuo to afford a crude product that was purified by flash chromatography on silica gel (solvent 20% EtOAc/petrol moving to 30% EtOAc/petrol) to afford the title compound (180 mg, 29%).

¹H NMR δ_{H} (CDCl₃, 300 MHz): 7.49 (d, J=1.8 Hz, 2H), 7.45 (t, J=1.8 Hz, 1H), 7.05 (s, 1H), 6.65 (br t, J=6.0 Hz, 1H), 4.03 (d, J=18 Hz, 1H), 3.96 (dq, J=6.0 and 9.0 Hz, 2H), 3.70 (d, J=18.0 Hz, 1H), 3.53 (s, 2H), 2.31 (s, 3H). ESI-MS 551.0 [MH]⁺.

### 3-[4-[5-(3,5-dichlorophenyl)-5-(trifluoromethyl)-4H-isoxazol-3-yl]-2-methyl-phenyl]sulfanylpyrrolidin-2-one 21

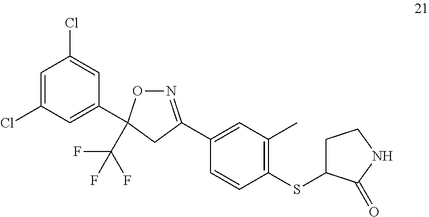 [SMILES: Cc1cc(C2=NOC(c3cc(Cl)cc(Cl)c3)(C(F)(F)F)C2)ccc1SC1CCNC1=O]

3-Sulfanylpyrrolidin-2-one was prepared according to the procedure provided in WO 2007139215. 5-(3,5-Dichlorophenyl)-3-(4-fluoro-3-methyl-phenyl)-5-(trifluoromethyl)-4H-isoxazole (254 mg, 0.65 mmol), 3-sulfanylpyrrolidin-2-one (114 mg, 0.97 mmol) and potassium carbonate (134 mg, 0.97 mmol) were mixed in DMF (2 mL) and the solution heated to 100° C. After 18 h, a further 3 equivalents of 3-sulfanylpyrrolidin-2-one (227 mg, 1.94 mmol) were added and the mixture heated for a further 24 h after which time the reaction was diluted with EtOAc (5 mL) and washed with H₂O (3×10mL). The organic fraction was dried over MgSO₄ and the solvent removed in vacuo to afford a crude product that was purified by flash chromatography on silica gel (10-40% EtOAc/petrol) to afford the title compound (80 mg, 12%).

¹H NMR δ_{H} (CDCl₃, 300 MHz): 7.50-7.34 (m, 6H), 6.11 (br s, 1H), 3.98 (d, J=17.1 Hz, 1H), 3.86 (dd, J=14.7 and 6.3 Hz, 1H), 3.73 (m, 2H), 2.60 (m, 1H), 2.34 (s, 3H), 2.10 (m, 1H); ESI-MS 489.1 [MH⁺].

### 3-[4-[5-(3,5-Dichlorophenyl)-5-(trifluoromethyl)-4H-isoxazol-3-yl]-2-methyl-phenyl]sulfinylpyrrolidin-2-one 22

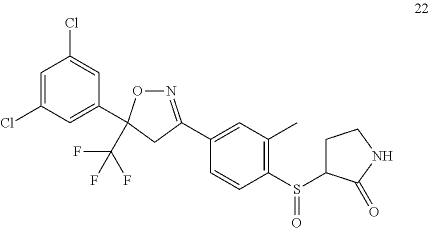 [SMILES: Cc1cc(C2=NOC(c3cc(Cl)cc(Cl)c3)(C(F)(F)F)C2)ccc1S(=O)C1CCNC1=O]

To a solution of 3-[4-[5-(3,5-Dichlorophenyl)-5-(trifluoromethyl)-4H-isoxazol-3-yl]-2-methyl-phenyl]sulfanylpyrrolidin-2-one (25 mg, 0.05 mmol) in DCM (1 mL) at 0° C. was added mCPBA (17 mg, 0.07 mmol) and the mixture was allowed to warm to rt over 16 h. H₂O (1 mL) was added, the organic layer separated and the aqueous layer further extracted with DCM (2×2 mL) before the combined organics were dried over MgSO₄ and the solvent removed in vacuo. The crude material was purified by flash chromatography on silica gel (solvent 70% EtOAc/hexane) to afford the title compound as a clear oil (8 mg, 31%).

¹H NMR δ_{H} (CDCl₃, 300 MHz): 7.92 (d, J=8.1 Hz, 1H), 7.70-7.55 (m, 2H), 7.51-7.50 (m, 2H), 7.43 (t, J=1.8 Hz, 1H), 6.57 (s, 1H), 4.10 (d, J=17.4 Hz, 1H), 3.71 (d, J=17.4 Hz, 1H), 3.59-3.47 (m, 2H), 3.45-3.35 (m, 1H), 2.76-2.63 (m, 1H), 2.45 (s, 3H), 1.91-1.78 (m, 1H). ESI-MS 505.1 [MH⁺].

### 3-[4-[5-(3,5-Dichlorophenyl)-5-(trifluoromethyl)-4H-isoxazol-3-yl]-2-methyl-phenyl]sulfonylpyrrolidin-2-one 23

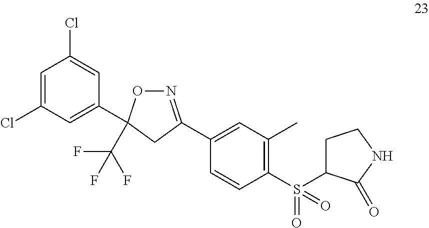 [SMILES: Cc1cc(C2=NOC(c3cc(Cl)cc(Cl)c3)(C(F)(F)F)C2)ccc1S(=O)(=O)C1CCNC1=O]

To a solution of 3-[4-[5-(3,5-Dichlorophenyl)-5-(trifluoromethyl)-4H-isoxazol-3-yl]-2-methyl-phenyl]sulfanylpyrrolidin-2-one (25 mg, 0.05 mmol) in DCM (1 mL) at 0° C. was added mCPBA (17 mg, 0.07 mmol) and the mixture was allowed to warm to rt over 16 h. H2O (1 mL) was added, the organic layer separated and the aqueous layer further extracted with DCM (2×2 mL) before the combined organics were dried over MgSO₄ and the solvent removed in vacuo. The crude material was purified by flash chromatography on silica gel (solvent 5% MeOH/DCM) to afford the title compound as a clear oil (9 mg, 35%).

¹H NMR δ_{H} (CDCl₃, 300 MHz): 8.02 (d, J=8.4 Hz, 1H), 7.69-7.60 (m, 2H), 7.53-7.48 (m, 2H), 7.44 (t, J=1.5 Hz, 1H), 6.08 (s, 1H), 4.09 (d, J=17.4 Hz, 1H), 3.93 (dd, J=9.9 and 3.3 Hz, 1H), 3.71 (d, J=17.4 Hz, 1H), 3.66 (dd, J=17.1 Hz and 7.8 Hz, 1H), 3.43 (td, J=2.9 and 9.2 Hz, 1H), 3.00-2.90 (m, 1H), 2.72 (s, 3H), 2.69-2.55 (m, 1H). ESI-MS 521.1 [MH⁺].

### 3-[4-[5-(3,5-Dichlorophenyl)-5-(trifluoromethyl)-4H-isoxazol-3-yl]-2-methyl-phenyl]sulfanyl-1-(2,2,2-trifluoroethyl)pyrrolidin-2-one 24

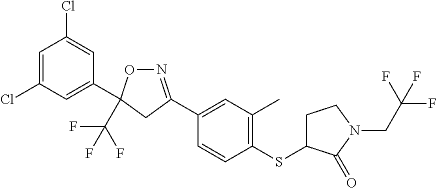 [SMILES: Cc1cc(C2=NOC(c3cc(Cl)cc(Cl)c3)(C(F)(F)F)C2)ccc1SC1CCN(CC(F)(F)F)C1=O]

To a solution of 3-[4-[5-(3,5-dichlorophenyl)-5-(trifluoromethyl)-4H-isoxazol-3-yl]-2-methyl-phenyl]sulfanylpyrrolidin-2-one 21 (25 mg, 0.055 mmol) in acetonitrile (1 mL) was added K₂CO₃ (18 mg, 0.06 mmol) and trifluoroethyl trifluoromethanesulfonate (8 μL, 0.06 mmol) and the mixture was heated to 50° C. for 16 h. Water (1 mL) and EtOAc (2 ml) were added, the layers separated and the aqueous phase was extracted with ethyl acetate (3×5 mL) before the combined organic fractions were dried over MgSO₄ and the solvent removed in vacuo. The residue was purified by flash chromatography on silica gel (solvent 10% EtOAc/hexane moving to 20% EtOAc/hexane) to afford the title compound (2 mg, 7%).

ESI-MS: 571.2, retention time 2.48 mins.

HRESI-MS: Found MNa⁺, 593.026693. C₂₃H₁₈Cl₂F₆N₂NaO₂S requires 593.026244.

The HPLC retention time was recorded under the following conditions:
Instruments: Agilent 1200 Series LC with diode array detector and Bruker HCT Ultra Ion Trap MS
Column: Phenomenex Luna 5u C18(2) 100 Å, 50×2.00 mm 5 micron LC column
Solvent: 5-90% gradient of acetonitrile in water (with 1% formic acid). Flow rate 1.2 mL/min.

### Example 4

### Pyranone Synthesis

### N-[2-Oxo-6-(3-pyridyl)pyran-3-yl]cyclopropanecarboxamide 25

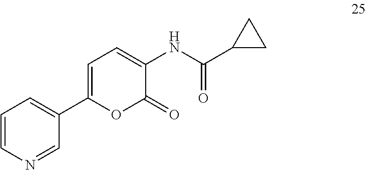 [SMILES: O=C(Nc1ccc(-c2cccnc2)oc1=O)C1CC1]

3-Amino-6-(3-pyridyl)pyran-2-one was prepared according to the procedure described in WO 2011/049150. Cyclopropanecarbonyl chloride (65 μL, 0.81 mmol) and triethylamine (0.13 mL, 0.96 mmol) were added to an ice-cold suspension of 3-amino-6-(3-pyridyl)pyran-2-one (138 mg, 0.74 mmol) in THF (5 mL). The reaction was stirred at ambient temperature for 90 minutes, after which time TLC showed complete consumption of the starting material. The reaction mixture was quenched with water (10 mL) and extracted with EtOAc (3×25 mL) before the combined organics were washed with brine (2×25 mL), dried over MgSO₄ and the solvent removed in vacuo to afford the title compound as a yellow solid (160 mg, 84%).

¹H NMR δ_{H} (DMSO-d6, 300 MHz): 10.08 (s, 1H), 9.02-9.01 (m, 1H), 8.63-8.62 (m, 1H), 8.24 (d, J=7.5 Hz, 1H), 8.19-8.15 (m, 1H), 7.55-7.51 (m, 1H), 7.24 (d, J=7.5 Hz, 1H), 2.32-2.24 (m, 1H), 0.82-0.80 (m, 4H). ESI-MS 257.0 [MH]⁺.

Compound 25 forms part of the prior art (WO2011/049150) and is included for reference purposes.

### N-[2-Oxo-6-(5-pyrimidyl)pyran-3-yl]cyclopropanecarboxamide 26

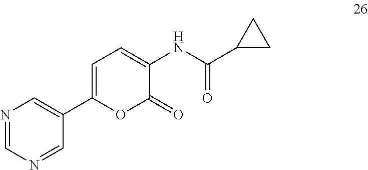 [SMILES: O=C(Nc1ccc(-c2cncnc2)oc1=O)C1CC1]

1-(5-Pyrimidyl)-3-(dimethylamino)-2-propene-1-one was prepared according to the procedure provided in WO 2013/007184. 2-(Cyclopropanecarboxamido)acetic acid was prepared according to the procedure provided in WO 2011049150. Acetic anhydride (2.70 mL, 28.56 mmol) was added to a mixture of 1-(5-pyrimidyl)-3-(dimethylamino)-2-propene-1-one (385 mg, 2.20 mmol) and 2-(cyclopropanecarboxamido)acetic acid (314 mg, 2.20) and the mixture was heated at 90° C. for 6 hours, after which time TLC showed complete consumption of the starting material. The volatiles were removed in vacuo before the crude material was purified by flash chromatography on silica gel (solvent EtOAc) to afford the title compound as a yellow solid (47 mg, 8%).

¹H NMR δ_{H} (DMSO-d6, 300 MHz): 10.12 (br, 1H), 9.32 (s, 1H), 9.20 (s, 2H), 8.26 (d, J=8.0 Hz, 1H), 7.33 (d, J=8.0 Hz, 1H), 2.31 (br, 1H), 0.84-0.82 (m, 4H) ppm. ESI-MS 258.0 [MH]⁺.

### Example 5

### Testing the Insecticidal/Acaricidal Activity of Compounds of the Invention

Aim

The compounds were screened for insecticidal efficacy against aphids, Myzus persicae, Cabbage moth, Mamestra brassicae, diamond black moths, Plutella xylostella, red spider mites, Tetranychus urticae, whitefly, Trialeurodes vaporariorum, in terms of knockdown and mortality.

Test system

Myzus persicae, Cabbage moth, Mamestra brassicae, diamond back moths, Plutella xylostella, red spider mites, Tetranychus urticae, whitefly, Trialeurodes vaporariorum, were obtained from specified laboratory cultures. Mixed sex and age aphids and mites, moth larvae and whitefly scale infested leaves were used in the experiments. Environmental conditions were closely monitored and recorded and were within the optimal range of the target species.

Test Treatments and Application

The compounds were screened at a range of five concentrations, diluted in DMSO. Some of the compounds were screened against aphids, whitefly and red spider mites and other compounds were screened against cabbage moth, and diamond black moths. A carrier only and an untreated control will also be conducted. Treatments were applied directly onto the insects within Petri dishes, using a potter tower.

Experimental Design

Twenty mites and ten aphids, scales and moth larvae were placed within a Petri dish, lined with a suitable leaf disc on a damp cotton wool pad. Scales were already on leaves. The Petri dishes were then sprayed using a potter tower. The number of knocked down and anthropods were assessed at 24 and 48 hours post treatment. Five replicates were be performed for each treatment, for each species.

The results are shown in Tables 1 and 2 (butenolide compounds), Tables 3 and 4 (pyridine compounds), Table 5 (oxazoline and isoxazoline compounds) and Table 6 (pyranone compounds) respectively.

For Tables 2, 4, 5 and 6, the compounds were diluted in DMSO containing 1% v/v Tween 20™. For Tables 2, 4, 5 and 6, the caterpillars were a mixture of Mamestra brassicae and Lacanobia oleracea.

A percentage control of from 80 to 100 is indicated by the letter A. A percentage control of from 60 to 79 is indicated by the letter B. A percentage control of from 40 to 59 is indicated by the letter C.

A percentage control of from 20 to 39 is indicated by the letter D. A percentage control of from 1 to 19 is indicated by the letter E.

TABLE 1

| | Dose | Aphids | | Spider Mites | |
|---|---|---|---|---|---|
| Compound | g/ha | 24 h | 48 h | 24 h | 48 h |
| 1 (flupyradifurone) | 400 | B | A | D | C |
| | 200 | C | A | D | D |
| | 100 | D | A | E | D |
| | 50 | D | A | D | D |
| | 25 | E | C | D | D |
| 3 | 400 | E | B | D | D |
| | 200 | E | C | D | D |
| | 100 | E | B | C | C |
| | 50 | 0% | C | D | D |
| | 25 | E | D | B | C |
| 6 | 400 | E | A | D | B |
| | 200 | E | D | B | B |
| | 100 | E | D | B | A |
| | 50 | E | C | B | A |
| | 25 | E | D | D | C |

TABLE 2
Average of % corrected affected

| Compound | | |
|---|---|---|
| 3 | 7 | 5 |

Hours post application

| | 24 | 48 | 24 | 48 | 24 | 48 |
|---|---|---|---|---|---|---|
| Aphids | | | | | | |
| 0.00625 (% | E | E | E | C | E | E |
| w/v) | | | | | | |
| 0.0125 | E | E | D | C | E | E |
| 0.025 | E | D | D | C | E | E |
| 0.05 | E | D | C | C | D | D |
| 0.1 | E | D | E | E | E | E |
| Caterpillars | | | | | | |
| 0.00625 | E | E | E | E | E | E |
| 0.0125 | E | E | E | 0 | E | E |
| 0.025 | E | E | E | E | E | E |
| 0.05 | E | E | 0 | 0 | E | E |
| 0.1 | E | D | E | E | E | D |

TABLE 3

| | | | | Cabbage | | spider | | Tomato | | |
|---|---|---|---|---|---|---|---|---|---|---|
| | Dose | Aphids | | moths | | mites | | moths | | Whitefly |
| Compound | g/ha | 24 h | 48 h | 24 h | 48 h | 24 h | 48 h | 24 h | 48 h | 72 h |
| 11 | 200 | B | A | E | E | C | C | E | E | |
| (sulfoxaflor) | | | | | | | | | | |
| | 100 | A | A | E | E | D | D | E | E | |
| | 50 | D | B | E | E | D | D | 0% | E | |
| | 25 | E | B | E | E | E | E | E | E | |
| | 12.5 | C | B | E | E | E | D | 0% | E | |
| 12 | 200 | E | D | | | B | A | | | E |
| | 100 | E | D | | | B | B | | | E |
| | 50 | E | C | | | C | C | | | E |
| | 25 | E | D | | | D | D | | | |
| | 12.5 | E | D | | | D | D | | | |
| 13 | 200 | D | C | | | D | B | | | E |
| | 100 | E | E | | | D | B | | | E |
| | 50 | E | D | | | D | B | | | E |
| | 25 | E | D | | | D | C | | | |
| | 12.5 | E | D | | | D | B | | | |
| 14 | 200 | E | D | | | B | B | | | |
| | 100 | E | D | | | C | B | | | |
| | 50 | E | D | | | D | C | | | |
| | 25 | E | E | | | C | C | | | |
| | 12.5 | E | D | | | D | C | | | |

TABLE 4
Average of % corrected affected

| Compound | | |
|---|---|---|
| 13 | 12 | 14 |

Hours post application

| | 24 | 48 | 24 | 48 | 24 | 48 |
|---|---|---|---|---|---|---|
| Aphids | | | | | | |
| 0.1 (% w/v) | D | D | D | C | D | D |
| Caterpillars | | | | | | |
| 0.00625 | | E | | E | | E |
| 0.0125 | | E | | E | | E |
| 0.025 | | E | | E | | E |
| 0.05 | | E | | E | | E |
| 0.1 | E | D | E | E | E | E |

The compounds of the invention have shown activity against a number of insect and arachnid pests. In particular, as shown in tables 1 and 3, compounds 6, 12, 13 and 14 all proved more active than the reference compound (flupyradifurone or sulfoxaflor respectively) against spider mites.

TABLE 5
Average of % corrected affected

| | Compound | | | | | | | | | |
|---|---|---|---|---|---|---|---|---|---|---|
| | 15 | | 18 | | 19 | | 20 | | 21 | |
| | Hours post application | | | | | | | | | |
| | 24 | 48 | 24 | 48 | 24 | 48 | 24 | 48 | 24 | 48 |
| Aphids | | | | | | | | | | |
| 0.000125 (% | E | E | E | E | D | C | E | E | D | D |
| w/v) | | | | | | | | | | |
| 0.00025 | E | E | E | E | E | D | E | E | E | E |
| 0.0005 | E | E | E | E | E | D | E | E | E | E |
| 0.001 | E | D | E | E | D | C | E | E | E | E |
| 0.002 | 0 | D | E | E | E | E | E | D | E | D |
| Caterpillars | | | | | | | | | | |
| 0.000125 | E | 0 | E | E | 0 | E | E | E | E | E |
| 0.00025 | E | E | 0 | E | 0 | E | E | E | E | E |
| 0.0005 | E | E | E | E | E | E | E | E | 0 | E |
| 0.001 | E | E | E | E | E | E | E | E | E | E |
| 0.002 | E | E | E | 0 | E | E | D | D | E | E |

The compounds of the invention have shown activity against insect pests. Compound 20 achieved more control over both aphids and caterpillars at 0.002% concentration than the reference compound 15.

TABLE 6
Average of % corrected affected

Compounds

| | | |
|---|---|---|
| 25 | 26 | 26 (2^{nd} batch) |

Hours post application

| | 24 | 48 | 24 | 48 | 24 | 48 |
|---|---|---|---|---|---|---|
| Aphids | | | | | | |
| 0.003125 (% | E | E | D | D | E | E |
| w/v) | | | | | | |
| 0.00625 | D | D | E | D | E | E |
| 0.0125 | D | D | E | E | 0 | E |
| 0.025 | C | B | E | E | E | E |
| 0.05 | E | C | E | E | E | E |
| Caterpillars | | | | | | |
| 0.003125 | 0 | E | E | E | E | E |
| 0.00625 | E | E | E | E | D | D |
| 0.0125 | E | E | E | E | E | E |
| 0.025 | E | D | E | E | E | E |
| 0.05 | E | E | D | D | E | E |

The compounds of the invention have shown activity against insect pests. Compound 26, achieved greater control than the reference compound 25 against caterpillars at 0.05%.

The compounds of the invention have shown some activity against insect and arachnid pests. Although this activity appears fairly low for certain compounds of the invention at present, the inventors expect that optimisation of the subtituent patterns for the compounds of the invention will result in improved activity, both in terms of absolute activity and when considered relative to prior art compounds. Given that at the concentrations tested even certain of the prior art compounds of known activity have achieved only modest control, it is also expected that retesting certain compounds of the invention at higher concentrations will result in increased control over the target species.

1. A compound of formula V: 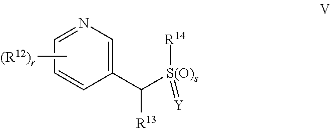
wherein Y is independently selected from N—S(O)₂R¹⁵, N—C(O)R¹⁵, NC(O)OR¹⁵, NC(O)NR^{15a}R¹⁵;
R¹² is independently at each occurrence selected from: C₁-C₄ alkyl, C₁-C₄-haloalkyl, halogen, nitro, OR¹⁶, SR¹⁶, cyano, C₂-C₄ alkeny, C₂-C₄ alkyny, C₃-C₆ cycloalkyl and NR¹⁶R¹⁶; R¹⁶ is independently at each occurrence selected from; H, C₁-C₄ alkyl, C(O)—C₁-C₄-alkyl and C₁-C₄ haloalkyl;
R¹³ is independently selected from: H, C₁-C₄ alkyl and C₁-C₄ haloalkyl;
R¹⁴ and R¹⁵ are each independently selected from: aryl, heteroaryl, C₁-C₄ alkyl and C₁-C₄ haloalkyl;
R^{15a} is independently selected from: H, C₁-C₄ alkyl and C₁-C₄ haloalkyl;
r is an integer independently selected from 0, 1, 2, 3 and 4;
s is an integer selected from 0 and 1;
wherein in any R¹²-R¹⁶ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a} and OR^{a};
wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;
or an agronomically acceptable salt or N-oxide thereof.

2. A compound of claim 1, wherein the compound of formula V is a compound of formula VIII:

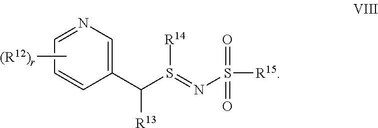

3. A compound of claim 1, wherein the compound of formula V is a compound of formula IX:

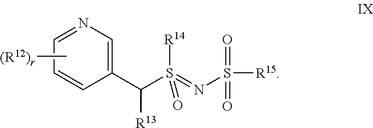

4. A compound of claim 1, wherein the compound of formula V is a compound of formula X:

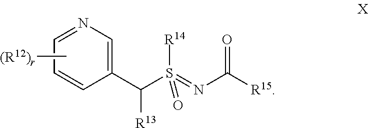

5. A compound of claim 1, wherein the compound of formula V is a compound of formula XI:

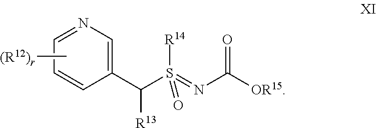

6. A compound of formula I: 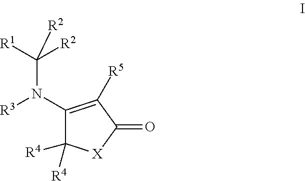
wherein X is independently selected from O or NR⁶;
R¹ is heteroaryl;
R², R⁴ and R⁷ are each independently at each occurrence selected from: H, halogen, C₁-C₄ alkyl and C₁-C₄ haloalkyl;
R³ is independently selected from: (CR⁷R⁷)ₙCO₂R⁸, (CR⁷R⁷)ₙCN, (CR⁷R⁷)ₙCOR⁸, (CR⁷R⁷)ₙCONR⁸R⁸ and (CR⁷CR⁷)CH(OR⁸)₂;
R⁵ is independently selected from H, halogen, C₁-C₄ alkyl and C₁-C₄ haloalkyl; or R³ and R⁵ together with the atoms to which they are attached form a heteroaromatic or heterocycloalkyl ring;
R⁶ and R⁸ are each independently selected from H, C₁-C₄ alkyl, C₃-C₆ cycloalkyl and C₁-C₄ haloalkyl;
n is an integer independently selected from 1, 2 and 3;
wherein in any R¹-R⁸ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a} and OR^{a};
wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;
or an agronomically acceptable salt or N-oxide thereof.

7. The compound of claim 6, wherein the compound of formula I is a compound of formula II: 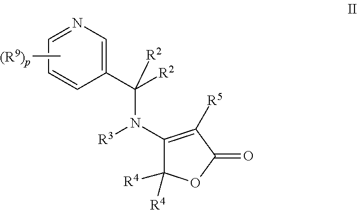
wherein R², R³, R⁴ and R⁵ are as described above for compounds of formula I and wherein R⁹ is independently at each occurrence selected from: C₁-C₄ alkyl, C₁-C₄-haloalkyl, halogen, nitro, OR¹⁰, SR¹⁰, cyano, C₂-C₄ alkenyl, C₂-C₄ alkynyl, C₃-C₆ cycloalkyl and NR¹⁰R¹⁰; R¹⁰ is independently at each occurrence selected from: H, C₁-C₄ alkyl, C(O)—C₁-C₄-alkyl and C₁-C₄ haloalkyl and p is an integer independently selected from 0, 1, 2, 3 and 4.

8. A compound of claim 6, wherein the compound of formula I is a compound of formula III: 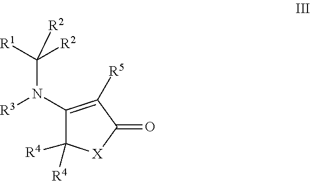
wherein R¹, R², R⁴ and X are as described above for compounds of formula I and wherein R³ is (CR⁷R⁷)ₙCO₂R⁸; and R⁵ is independently selected from H, halogen C₁-C₄ alkyl and C₁-C₄ haloalkyl.

9. A compound of claim 6, wherein the compound of formula I is a compound of formula IV: 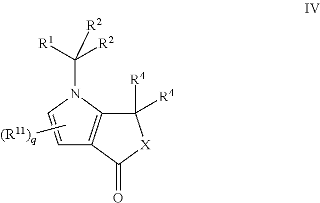
wherein R¹, R², R⁴ and X are as described above for compounds of formula I and wherein R¹¹ is independently at each occurrence elected from: C₁-C₄ alkyl and C₁-C₄ haloalkyl, halogen, nitro, OR¹⁰, SR¹⁰, cyano, C₂-C₄ alkyny, C₃-C₆ cycloalkyl and NR¹⁰R¹⁰; R¹⁰ is independently at each occurrence selected from: H, C₁-C₄ alkyl, CO)—C₁-C₄ alkyl and C₁-C₄ haloalkyl and q is an integer selected from 0, 1 and 2.

10. A compound of formula XII: 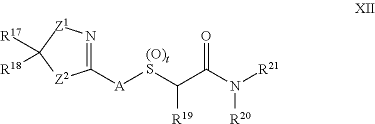
wherein Z¹ and Z² are each selected from O and CH₂; with the proviso that one of Z¹ and Z² is O and the other is CH₂;
A is independently selected from a phenyl group, a pyridyl group, a pyridazyl group, a pyrimidyl group, a pyrazyl group and a thiophenyl group;
R¹⁷ is aryl;
R¹⁸ is independently selected from H, halogen, C₁-C₄ alkyl and C₁-C₄ haloalkyl;
R¹⁹ is independently selected from H, halogen, C₁-C₄ alkyl, C₃-C₆-cycloalkyl and C₁-C₄ haloalkyl;
R²⁰ and R²¹ are each independently selected from H, C₁-C₄ alkyl, C₃-C₆-cycloalkyl and C₁-C₄ haloalkyl; or wherein R¹⁹ and R²⁰, together with the atoms to which they are attached form a 5- or 6-membered lactam ring; with the proviso that, if Z¹ is O and R¹⁹ and R²⁰ do not, together with the atoms to which they are attached, form a lactam ring, A is thiophenyl;
t is an integer independently selected from 0, 1 and 2;
wherein in any R¹⁷-R²¹ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃Ra; SO₂R^{a}; SO₂NR^{a}R^{a}; CO₂R^{a}; C(O)R^{a}; CONR^{a}R^{a}; CH₂NR^{a}R^{a}; CH₂OR^{a} and OR^{a};
wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;
or an agronomically acceptable salt or N-oxide thereof.

11. A compound of formula XVI: 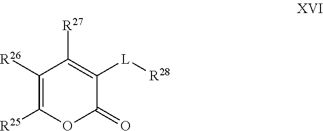
wherein L is independently selected from —NR²⁹—C(O)— and —N═CR³⁰—;
R²⁵ is independently selected from pyridyl, pyrimidyl, pyrazyl and pyridazyl;
R²⁶ and R²⁷ are each independently selected from H, halogen, C₁-C₄ alkyl and C₁-C₄ haloalkyl;
R²⁹ is independently selected from H, OR³¹, C₁-C₄ alkyl, C₃-C₆ cycloalkyl and C₁-C₄ haloalkyl;
R²⁸, R³⁰ and R³¹ are each independently selected from H, C₁-C₄ alkyl, C₃-C₆ cycloalkyl and C₁-C₄ haloalkyl; or R²⁷ and R²⁸, together with the atoms to which they are attached, form a 5- or 6-membered lactam ring;
with the proviso that if R²⁵ is pyridyl, L is —NR²⁹—C(O)— and R²⁷ and R²⁸ do not, together with the atoms to which they are attached, form a 5- or 6-membered lactam ring, R²⁹ is OR³¹;
wherein in any R²⁵-R³¹ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}; CO₂R^{a}; C(O)R^{a}; CONR^{a}R^{a}; CH₂NR^{a}R^{a}; CH₂OR^{a} and OR^{a};
wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;
or an agronomically acceptable salt or N-oxide thereof.

12. A method for controlling insect and arachnid pests of plants, the method comprising applying an agronomically effective and substantially non-phytotoxic (to the crop plant) quantity of a compound of claim 1 or a compound of formula Vb to the seeds of the plants, to the plants themselves or to the area where it is intended that the plants will grow 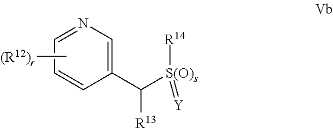
wherein Y is independently selected from O and N—CN;
R¹² is independently at each occurrence selected from: C₁-C₄ alkyl, C₁-C₄-haloalkyl, halogen, nitro, OR¹⁶, SR¹⁶, cyano, C₂-C₄ alkeny, C₂-C₄ alkyny, C₃-C₆ cycloalkyl and NR¹⁶R¹⁶; R¹⁶ is independently at each occurrence selected from; H, C₁-C₄ alkyl, C(O)—C₁-C₄-alkyl and C₁-C₄ haloalkyl;
R¹³ is independently selected from: H, C₁-C₄ alkyl and C₁-C₄ haloalkyl;
R¹⁴ and R¹⁵ are each independently selected from: aryl, heteroaryl, C₃-C₈-cycloalkyl, C₃-C₈-heterocycloalkyl, C₁-C₄ alkyl and C₁-C₄ haloalkyl;
r is an integer independently selected from 0, 1, 2, 3 and 4;
s is an integer selected from 0 and 1; with the proviso that if Y is N—CN, s is 0;
wherein in any R¹²-R¹⁶ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}, CO₂R^{a}, C(O)R^{a}; CONR^{a}R^{a}; CH₂NR^{a}R^{a}; CH₂OR^{a} and OR^{a};
wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;
or an agronomically acceptable salt or N-oxide thereof.

13. An insecticidal or acaricidal composition comprising an effective and non-phytotoxic amount of an active compound of claim 1 or a compound of formula Vb 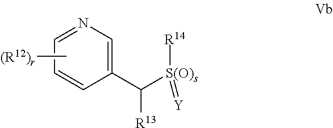
wherein Y is independently selected from O and N—CN;
R¹² is independently at each occurrence selected from: C₁-C₄ alkyl, C₁-C₄-haloalkyl, halogen, nitro, OR¹⁶, SR¹⁶, cyano, C₂-C₄ alkeny, C₂-C₄ alkyny, C₃-C₆ cycloalkyl and NR¹⁶R¹⁶; R¹⁶ is independently at each occurrence selected from; H, C₁-C₄ alkyl, C(O)—C₁-C₄-alkyl and C₁-C₄ haloalkyl;
R¹³ is independently selected from: H, C₁-C₄ alkyl and C₁-C₄ haloalkyl;
R¹⁴ and R¹⁵ are each independently selected from: aryl, heteroaryl, C₃-C₈-cycloalkyl, C₃-C₈-heterocycloalkyl, C₁-C₄ alkyl and C₁-C₄ haloalkyl;
r is an integer independently selected from 0, 1, 2, 3 and 4;
s is an integer selected from 0 and 1; with the proviso that if Y is N—CN, s is 0;
wherein in any R¹²-R¹⁶ group which contains an alkyl, haloalkyl, cycloalkyl, heterocycloalkyl, aryl (including phenyl, biphenyl and naphthyl) or heteroaryl group, that alkyl, haloalkyl, cycloalkyl, heterocycloalkyl, aryl or heteroaryl group is optionally substituted, where chemically possible, by 1 to 4 substituents which are each independently selected at each occurrence from the group consisting of: oxo; ═NR^{a}; ═NOR^{a}; R^{a}; halo; nitro; cyano; NR^{a}R^{a}; SO₃R^{a}; SO₂R^{a}; SO₂NR^{a}R^{a}, CO₂R^{a}, C(O)R^{a}; CONR^{a}R^{a}; CH₂NR^{a}R^{a}; CH₂OR^{a} and OR^{a};
wherein R^{a} is selected from H, C₁-C₄ alkyl and C₁-C₄ haloalkyl; and wherein, in the case of an aryl group or heteroaryl group, any two of these substituents (e.g. NR^{a}R^{a}, OR^{a}, SR^{a}, R^{a}) when present on neighbouring atoms in the aryl or heteroaryl group may, where chemically possible, together with the atoms to which they are attached form a ring which is fused to the aryl or heteroaryl group;
or an agronomically acceptable salt or N-oxide thereof.
